(19) United States
(12) Patent Application Publication
(10) Pub. No.: US 2025/0114160 A1
(43) Pub. Date: Apr. 10, 2025

(54) SURGICAL INSTRUMENTS

(71) Applicant: 4S Medical, LLC, Rocky River, OH (US)

(72) Inventor: Anton G. C. Milo, Akron, OH (US)

(73) Assignee: 4S Medical, LLC, Rocky River, OH (US)

(21) Appl. No.: 18/899,712

(22) Filed: Sep. 27, 2024

Related U.S. Application Data
(60) Provisional application No. 63/587,872, filed on Oct. 4, 2023.

(51) Int. Cl. A61B 50/31; A61B 1/247; A61B 1/32; A61B 17/00; A61B 17/12; A61B 17/28; A61B 17/3207; A61B 50/00; A61B 90/00

(52) U.S. Cl. CPC A61B 50/31; A61B 1/247; A61B 1/32; A61B 17/2816; A61B 17/282; A61B 17/2841; A61B 17/320708; A61B 2017/00438; A61B 2017/00477; A61B 2017/00738; A61B 2017/12004; A61B 2050/0084; A61B 2090/0817; A61B 2217/005

(57) ABSTRACT

Exemplary embodiments of surgical instruments and packs or sets of surgical instruments are disclosed herein. An exemplary surgical instrument is a two-part surgical instrument that includes a first shank. The first shank has a first ring handle at a first end and a first jaw portion on the other end and a shaft and one or more retainers. The exemplary surgical instrument includes a second shank. The second shank has a second ring handle at a first end and a second jaw portion on the other end and an opening configured to receive the shaft. The first shank is connected to the second shank by sliding the opening of the second shank over the shaft of the first shank and the first shank is disconnected from the second shank by pulling the second shank away from the first shank.

### RELATED APPLICATIONS

This application claims the benefits of and priority to U.S. Provisional Patent Application Ser. No. 63/587,872, titled SURGICAL INSTRUMENTS, and filed on Oct. 4, 2023, which is incorporated herein by reference in its entirety.

### TECHNICAL FIELD

The present invention relates generally to surgical instruments and more particularly to multi-function surgical instruments, hemostats, forceps, mouth gags, palate retractors, and sets of surgical instruments and expandable sets of surgical instruments.

### BACKGROUND OF THE INVENTION

Surgical instruments are typically provided in a sterile package. The sterile package may contain an individual surgical instrument or may contain a selected number of surgical instruments that are grouped together as surgical sets or trays for a surgery. The surgical instruments are typically made of surgical stainless steel. The surgical instruments are expensive. Often there are upwards of 30 surgical instruments combined and packaged by the sterile processing department in a surgical tray for each surgical procedure. A surgical assistant or nurse typically opens the package, lays out the surgical instruments and organizes them on a stand or table based on the type of surgery and/or the surgeon's preferences. Laying out and organizing the surgical instruments takes up valuable operating room (“OR”) time.

After use, the surgical instruments are collected, loaded on a cart, sprayed down and covered in sterile drapes. Then the cart is rolled out of the OR and taken back to the sterile processing department. This process consumes valuable OR time. After removal from the OR, the surgical instruments are cleaned, inspected for blood and/or tissue and cracks, repaired (if possible), sterilized and repackaged for a future surgical procedure. Furthermore, the mouth gag needs to be disassembled, which involves removing screws, springs and the silicon guard. Repairing the surgical instruments is time consuming. Replacing the surgical instruments is expensive. Maintaining spare inventory to replace entire instruments and parts of the surgical instruments is expensive. Cleaning, inspecting, repairing, sterilizing, reassembling the mouth gag and replacing the silicone guard, and packaging the surgical instruments is labor intensive and is very costly. Furthermore, failure to remove all of the blood and/or tissue from the surgical instrument may also result in a hospital quired infections and/or contaminated surgical instrument or tray of surgical instruments.

In addition, the surgical instrument may fail during use, if for example, a small crack was missed during the inspection. Failure of a surgical instrument increases OR time while a new instrument is obtained and may result in injury to the patient. In addition, many of the prior art surgical instruments contain small parts, such as, for example, rivets, springs, screws, and the like. Occasionally, one or more of these small parts separates from the surgical instrument. When this happens during a surgical procedure, the small part may end up in the patient's throat or lungs. Not only does this provide a safety concern and increase OR time, but it may require an x-ray to locate the part so that it can be removed.

Accordingly, there exists a need for surgical instruments, and/or sets of surgical instruments that do not suffer from the deficiencies identified above.

### SUMMARY

Exemplary embodiments of surgical instruments and packs or sets of surgical instruments are disclosed herein. An exemplary surgical instrument is a two-part surgical instrument that includes a first shank. The first shank has a first ring handle at a first end, a first jaw portion on the other end, and a shaft and one or more retainers located at a pivot point. The exemplary surgical instrument includes a second shank. The second shank has a second ring handle at a first end, a second jaw portion on the other end, and an opening located at the pivot point and configured to receive the shaft. The first shank is connected to the second shank by sliding the opening of the second shank over the shaft of the first shank and the first shank is disconnected from the second shank by pulling the second shank away from the first shank.

Another exemplary surgical instrument is a hemostat that includes a first shank having a ring handle, a first portion of a ratchet mechanism on a first end, a first portion of a jaw on a second end, and a first portion of a releasable connector at a pivot point. The exemplary hemostat includes a second shank having a ring handle and a second portion of a ratchet mechanism on a first end, a second portion of a jaw on a second end, and a second portion of a releasable connector at a pivot point. The releasable connector is configured to couple the first shank to the second shank and to uncouple the first shank from the second shank without the use of tools.

Another exemplary hemostat includes a first shank having a ring handle, a first portion of a ratchet mechanism on a first end, a first portion of a jaw on a second end and a first portion of a releasable connector at a pivot point. The hemostat includes a second shank having a ring handle and a second portion of a ratchet mechanism on a first end, a second portion of a jaw on a second end and a second portion of a releasable connector at a pivot point. The releasable connector is configured to couple the first shank to the second shank and to uncouple the first shank from the second shank without the use of tools. In the connected configuration the first jaw and the second jaw are configured to meet and form a working surface of the jaw. A first part of the working surface extends in a first direction and a second part of the working surface extends from the end of the first part along a second direction that is different than the first direction. A first set of serrations are located proximate an end of the second part of the working surface. A smooth flat section located proximate the first set of serrations.

Another exemplary surgical instrument is a modified Allis forceps. The Allis forceps include a first shank having a ring handle and a first portion of a ratchet mechanism on a first end, a first portion of a jaw on a second end, a first projection extending from an end of the first portion of the jaw, and a first portion of a releasable connector at a pivot point. The Allis forceps include a second shank having a ring handle and a second portion of a ratchet mechanism on a first end, a second portion of a jaw on a second end, a second projection extending from an end of the second portion of the jaw and a second portion of a releasable connector at a pivot point. The releasable connector is configured to couple the first shank to the second shank and to uncouple the first shank from the second shank without the use of tools. When the first shank and second shank are in the coupled position, the first projection and the second projection extend toward one another.

Another exemplary surgical instrument, that is, for example, a modified forceps, includes a first shank having a ring handle and a first portion of a ratchet mechanism on a first end, a first portion of a jaw on a second end, a first projection extending from an end of the first portion of the jaw, and a first portion of a releasable connector at a pivot point. The first portion of the jaw extends along a first direction and a second portion of the jaw extends along a second direction that is different than the first direction. The modified Allis forceps include a second shank having a ring handle and a second portion of a ratchet mechanism on a first end, a second portion of a jaw on a second end, a second projection extending from an end of the second portion of the jaw and a second portion of a releasable connector at a pivot point. The second portion of the jaw extends along a first direction and a second portion of the jaw extends along a second direction that is different than the first direction. The releasable connector is configured to couple the first shank to the second shank and to uncouple the first shank from the second shank without the use of tools. When the first shank and second shank are in the coupled position, the first projection and the second projection extend toward one another.

Another exemplary surgical instrument is a palate retractor. The palate retractor includes a body, a mouth gag connection member on a first end of the body, a grip member, a palate engagement member located on a second end of the body, and a pair of wings extending outward from the body proximate the palate engagement member.

Another exemplary multi-functional surgical instrument is a multi-function tri-view surgical instrument. The multi-function tri-view surgical instrument includes a body, a spatula shaped elevator located at a first end of the polymeric body, and a mirror head located at a second end of the body. The mirror head extends from the body at an angle. The angle is greater than zero degrees. A retractor, such as a pillar retractor, extends outward from a back of the mirror head.

Another exemplary multi-functional surgical instrument is a curette. The curette includes a handle and a head. The head includes a pair of forks and a blade. The blade comprises a first cutting edge and a second cutting edge. The first and second cutting edges are both sharp enough to cut tissue. The first cutting edge is on an opposite side of the second cutting edge. When moved in a first direction, the first cutting edge is positioned to cut tissue, and when moved in a second direction, the second cutting edge is positioned to cut tissue.

The exemplary surgical instruments are combinable into preorganized packs of surgical instruments. An exemplary preorganized pack of surgical instruments includes at least one hemostat and at least one forceps. Wherein one of the hemostat and the forceps are readily separatable into two pieces.

Another exemplary multi-functional surgical instrument includes a first shank. The first shank has a ring handle at a first end and a portion of a jaw on the other. The exemplary multi-functional surgical instrument further includes a second shank. The second shank has a ring handle at a first end and a portion of a jaw on the other. The exemplary multi-functional surgical instrument includes a releasable connector. The releasable connector is configured so that the first shank and second shank may be separated from one another and connected to one another without the use of tools.

Another exemplary surgical instrument is a ring blade holder for a mouth gag. The ring blade holder has a unitary body. The unitary body includes a ring member, wherein the ring member forms a working opening. The unitary body includes a channel located in the body for receiving a spine of a tongue blade and a latching member formed in the body. The body includes a channel through a surface of the body located at least partially around the latching member. A flexible resilient hinge is formed at least in part by the surface of the body proximate an end of the channel. The latching member extends below an upper surface of the channel. Movement of the latching member toward the upper surface of the channel causes a biasing force in the flexible resilient hinge that biases the latching member away from the upper surface of the channel.

Another exemplary pack of surgical instruments includes a plurality of surgical instruments. At least one of the plurality of surgical instruments includes a first body portion having a first part of a releasable connector and a second body portion having a second part of the releasable connector. The first body portion and the second body portion may be connected together and separated from one another.

Another exemplary surgical instrument includes a first part having a first shank, a first ring handle, a first working end and a first portion of a releasable connector. The surgical instrument includes a second part having a second shank, a second ring handle, a second working end and a second prat of a releasable connector. The first working end has a different configuration than the second working end. The first portion of a releasable connector connects to the second portion of a releasable connector to couple the first part to the second part in a coupled position. In the coupled position, movement of the first ring handle away from the second ring handle causes the first working end and the second working end to move away from one another. Movement of the first ring handle toward the second ring handle causes the first working end to move toward the second working end. Another exemplary surgical instrument comprises a suction tongue blade having a spine, a suction connector, a hollow suction pathway, an engagement surface, and suction inlet; wherein the suction inlet is located along a centerline of the engagement surface.

Another exemplary surgical instrument includes a first part having a first shank, a first ring handle, a first working end and a first portion of a releasable connector; and a second part having a second shank, a second ring handle, a second working end and a second prat of a releasable connector. The first working end has a different configuration than the second working end. The first portion of a releasable connector connects to the second portion of a releasable connector to couple the first part to the second part in a coupled position. In the coupled position, movement of the first ring handle away from the second ring handle causes the first working end and the second working end to move away from one another. Movement of the first ring handle toward the second ring handle causes the first working end to move toward the second working end.

Another exemplary preorganized pack of surgical instruments incudes a package, at least one hemostat and at least one forceps. Wherein at least one of the hemostat and the forceps are readily separatable into two pieces and wherein the at least one hemostat and the at least one forceps are devoid of rivets, screws or bolts.

Another exemplary preorganized pack of surgical instruments has a package made of a semi-rigid material. The package includes a plurality of tabs. The plurality of tabs are cut out from a portion of the semi-rigid material. One or more openings are located in the plurality of tabs. The plurality of tabs are bent away from the semi-rigid material and the one or more openings are configured to secure at least a portion of a surgical instrument.

Another exemplary preorganized pack of surgical instruments has a package made of a semi-rigid material. The package includes a plurality of tabs. The plurality of tabs are cut out from a portion of the semi-rigid material. One or more openings are located in the plurality of tabs. The plurality of tabs are bent away from the semi-rigid material. The plurality of tabs are configured such that a first surgical instrument is secured by one or more openings; and a second surgical instrument is secured by one or more different openings; and at least a portion of the second surgical instrument is located above the first surgical instrument.

Exemplary methodologies for providing packs of surgical instruments are provide herein. An exemplary methodology for providing a pack of surgical instruments include providing a package substrate, providing a plurality of surgical instruments and configuring retainers to retain the plurality of surgical instruments. Configuring the retainers to retain the plurality of surgical instruments includes providing one or more cut-outs in the package substrate that are configured to be bent outward from the package to retain the plurality of surgical instruments. The exemplary methodology incudes arranging the plurality of surgical instruments, sealing the pack of surgical instruments and sterilizing the plurality of surgical instruments.

Another exemplary methodology for providing a pack of surgical instruments comprises providing a package substrate and providing a plurality of surgical instruments. At least one of the surgical instruments comprises one of a hemostat and a forceps, and the at least one of the hemostat and the forceps are separable into two parts by rotating the two parts to a selected position and moving the two parts away from one another. The methodology further comprises configuring retainers to retain the plurality of surgical instruments. Configuring the retainers to retain the plurality of surgical instruments includes providing one or more cut-outs in the package substrate that are configured to be bent outward from the package to retain the plurality of surgical instruments. The methodology includes arranging the plurality of surgical instruments, sealing the pack of surgical instruments and sterilizing the plurality of surgical instruments.

Another exemplary methodology for providing a pack of surgical instruments includes providing a package substrate, providing a plurality of surgical instruments, and configuring retainers to retain the plurality of surgical instruments. Configuring the retainers to retain the plurality of surgical instruments includes providing one or more cut-outs in the package substrate that are configured to be bent outward from the package to retain the plurality of surgical instruments. The methodology includes arranging the plurality of surgical instruments. Arranging the plurality of surgical instruments includes locating at least one the plurality of surgical instruments on top of at least one other of the plurality of surgical instruments. The methodology further includes sealing the pack of surgical instruments and sterilizing the plurality of surgical instruments.

An exemplary methodology for providing a pack of surgical instruments includes providing a package substrate and providing a plurality of surgical instruments. At least one of the surgical instruments includes a multi-function surgical instrument that separates into two separate parts. The methodology further includes configuring retainers to retain the plurality of surgical instruments, arranging the plurality of surgical instruments, sealing the pack of surgical instruments and sterilizing the plurality of surgical instruments.

Yet another exemplary methodology for providing a pack of surgical instruments includes providing a package substrate, providing a plurality of surgical instruments. At least one of the surgical instruments comprises a multi-function surgical instrument with a first end that is configured to perform a first function and a second end opposite the first end that is configured to perform a second function. The methodology further includes configuring retainers to retain the plurality of surgical instruments, arranging the plurality of surgical instruments, sealing the pack of surgical instruments and sterilizing the plurality of surgical instruments.

Exemplary embodiments of the surgical instruments disclosed herein are single-use surgical instruments. The single-use surgical instruments are made of a polymer and are disposed of after a single use. The single-use surgical instruments are non-metallic. The single-use surgical instruments may be devoid of metallic springs. The single-use surgical instruments may be devoid of screws. The single-use surgical instruments may be devoid of small parts. Small parts are parts smaller than, for example, 10 millimeters.

### BRIEF DESCRIPTION OF THE DRAWINGS

These and other features and advantages of the present invention will become better understood with regard to the following description and accompanying drawings in which:

FIG. 1 is an exemplary tray or group of surgical instruments;

FIGS. 1A and 1B are an exemplary pack of surgical instruments;

FIG. 2 is a prospective view of a first exemplary multi-function hemostat with a needle driver;

FIG. 3 is a back view of the hemostat of FIG. 2;

FIG. 4 is a front view of the hemostat of FIG. 2;

FIG. 5 is a side view of the hemostat of FIG. 2;

FIG. 5A is an enlarged view of the jaws and connector of the hemostat of FIG. 2;

FIG. 6 is a prospective view of the hemostat of FIG. 2 separated into two pieces;

FIG. 7 is a front view of the hemostat of FIG. 2 separated into two pieces;

FIG. 8 is an enlarged view of the jaws of the hemostat of FIG. 2;

FIG. 9 is a prospective view of an exemplary embodiment of a first multi-function forceps;

FIG. 10 is a front view of the forceps of FIG. 9;

FIGS. 11A, 11B are enlarged views of the jaws of the forceps of FIG. 9;

FIG. 12 is a prospective view of an exemplary mouth gag having an exemplary ring blade holder and an exemplary suction tongue blade;

FIG. 13 is a prospective view of an exemplary unitary body ring blade holder;

FIG. 14 is a top view of the unitary body ring blade holder of FIG. 13;

FIG. 15 is a bottom view of the unitary body ring blade holder of FIG. 13;

FIG. 16 is a prospective view of an exemplary suction tongue blade;

FIG. 17 is a front view of the suction tongue blade of FIG. 16;

FIG. 18 is a back view of the suction tongue blade of FIG. 16;

FIG. 19 is a prospective view of an exemplary palate retractor;

FIG. 20 is another prospective view of the palate retractor of FIG. 19;

FIG. 21 is a bottom view of the exemplary palate retractor of FIG. 19;

FIG. 22 is a top view of the exemplary palate retractor of FIG. 19;

FIG. 22A is a prospective view of the palate retractor of FIG. 19 attached to a mouth gag;

FIG. 22B is a side view of the palate retractor of FIG. 19 attached to a mouth gag;

FIG. 23 is a prospective view of an exemplary multi-function tri-view surgical instrument that includes a mirror, a tissue retractor and spatula retractor or elevator;

FIG. 24 is a rear view of the multi-function tri-view surgical instrument that includes a mirror, tissue retractor and spatula retractor or elevator of FIG. 23;

FIG. 25 is a front view of the multi-function tri-view surgical instrument that includes a mirror, tissue retractor and spatula retractor or elevator of FIG. 23;

FIG. 26 is a side view of the multi-function tri-view surgical instrument that includes a mirror, tissue retractor and spatula retractor or elevator of FIG. 23;

FIG. 27 is a prospective view of an exemplary curette;

FIG. 28 is an enlarged prospective view of the head of the curette of FIG. 27;

FIG. 29 a prospective view of another exemplary multi-function hemostat with a needle driver;

FIG. 30 is a front view of the hemostat of FIG. 29;

FIG. 31 is a side view of the hemostat of FIG. 29;

FIG. 32 is a prospective view of a first half of the hemostat of FIG. 29;

FIG. 32A is an enlarged view of the jaw of the first half of the hemostat of FIG. 32;

FIG. 33 is a prospective view of a second half of the hemostat of FIG. 29;

FIG. 33A is an enlarged view of the jaw of the second half of the hemostat of FIG. 32;

FIG. 34 is an enlarged view of the jaws of the hemostat;

FIG. 35 is a prospective view of another exemplary multi-function modified Allis forceps;

FIG. 36 is a second prospective view of the multi-function Allis forceps of FIG. 35;

FIG. 36A is an enlarged view of a portion of a jaw of the multi-function Allis forceps;

FIG. 37 is a front view of the multi-function allis forceps of FIG. 35;

FIG. 37A is an enlarged view of a portion of a jaw of the multi-function Allis forceps;

FIG. 38 is an exemplary embodiment of hybrid surgical instrument;

FIG. 39 through FIG. 41 are exemplary working ends for hybrid surgical instruments;

FIGS. 42-44 illustrate exemplary contact points for selected hybrid surgical instruments;

FIGS. 45 through 45C disclose another exemplary embodiment of a multi-functional forceps surgical tool; and

FIG. 46 is an exemplary methodology for providing a sterilized pack of surgical instruments.

### DETAILED DESCRIPTION

The following includes definitions of exemplary terms used throughout the disclosure. Both singular and plural forms of all terms fall within each meaning. Except where noted otherwise, capitalized and non-capitalized forms of all terms fall within each meaning.

The terms readily disarticulated, readily separated, easily separated, separatable and the like mean that the surgical instrument may be easily disassembled and reassembled by the surgeon, a surgical nurse or physician's assistant during a surgery. Preferably, the surgical instrument may be disassembled and reassembled without the use of a tool. The term releasable connector does not include permanent connectors that cannot be easily connected and disconnected. Examples of permanent connectors are rivets, screws (that require a screwdriver to remove), bolts/nuts, and the like.

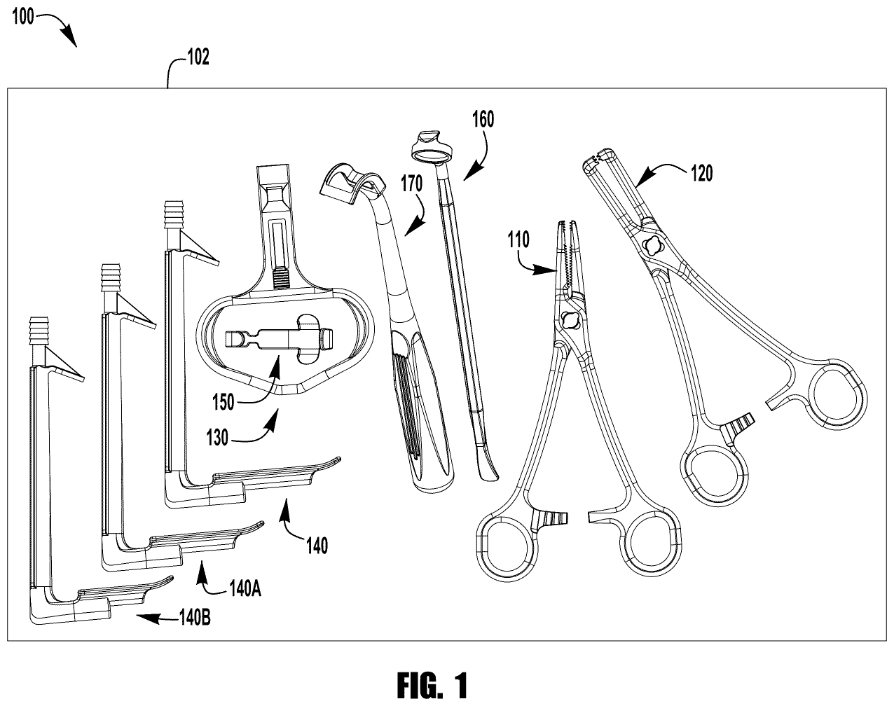

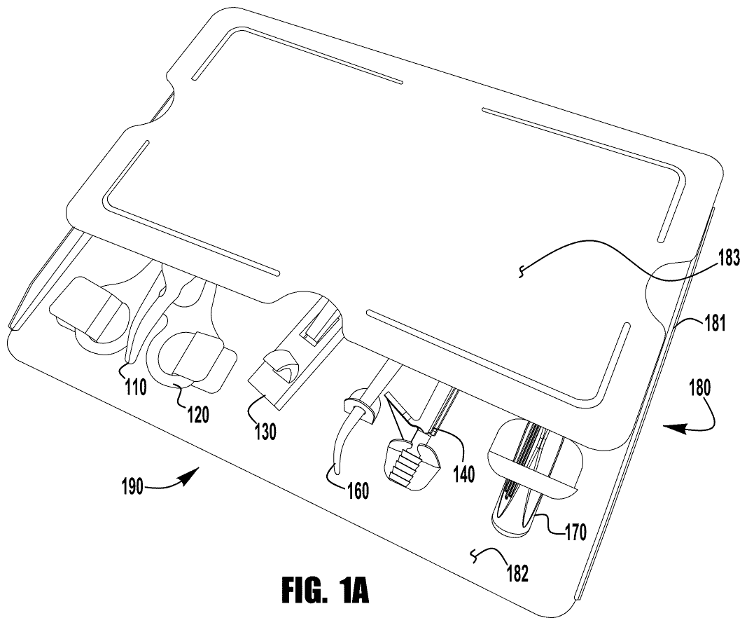

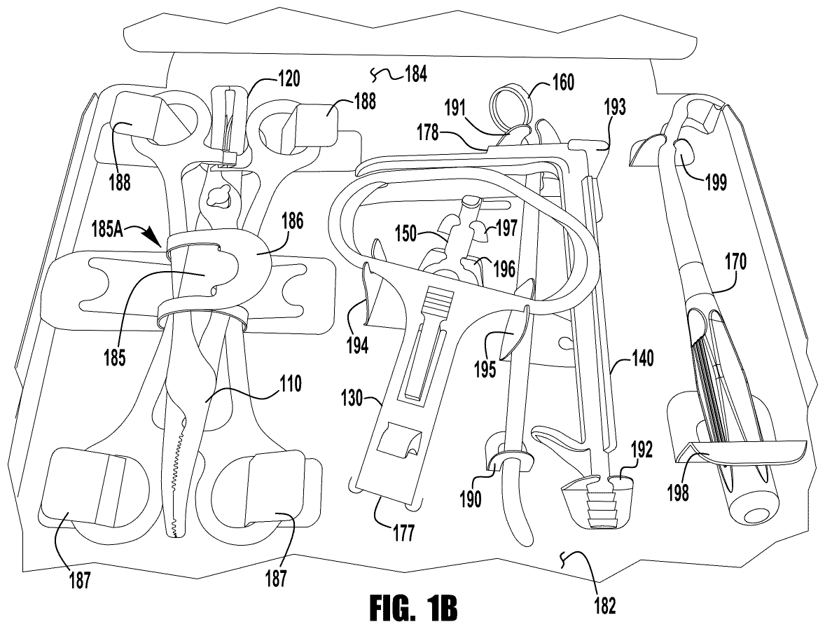

FIG. 1 is an exemplary embodiment of tray 102 of surgical instruments100. As discussed in more detail below, many of the surgical instruments 100 described herein are multi-functional and are configured to be used to perform multiple different tasks, each of which are conventionally performed by a single instrument. This exemplary tray 102 of surgical instruments 100 is configured for oral and/or pharyngeal surgeries, such as, for example, tonsil excision/removal. The exemplary surgical instruments 100 and concepts described and claimed herein are applicable to surgical instruments used in other types of surgeries.

The exemplary tray 102 of surgical instruments 100 includes a tray 102 and seven surgical instruments 100. The tray 102 may be a conventional tray that includes, for example, a bottom and sides. However, tray 102 may simply be a package for holding the surgical instruments. Tray 102 includes a multi-functional hemostat 110, a multi-functional modified Allis forceps 120, a ring blade holder 130, a plurality of different sized suction tongue blades 140, 140A and 140B, a palate retractor 150, a multi-function tri-view instrument 160 that includes a mirror, a tissue retractor and spatula retractor or elevator, and a curette 170.

While the tray 100 includes three different sized suction tongue blades 140, 140A, 140B, the tray 100 may contain fewer tongue blades. For example, an individual tray 102 may include suction tongue blade 140 for use with large individuals, e.g. adults. Another individual tray 102 may include suction tongue blade 140A for use with medium sized individuals. Similarly, another tray 102 may include suction tongue blade 140B for use with small sized individuals, e.g. children. While tray 102 is described with the selected set of surgical instruments, tray 102 may be configured with sets of different surgical instruments.

FIGS. 1A and 1B illustrate an exemplary pack 180 of surgical instruments 190. Exemplary pack 180 of surgical instruments includes a package 181. Package 181 may be made out of any material, such as, for example, cardboard, plastic, or the like. Preferably the package is made of a semi-rigid material. Preferably package 181 is made of a recyclable material. Package 181 includes a base 182, an optional cover 183, an optional back 184, and optional sides. Cover 183 may include a smooth surface that may include printed material, such as, for example, a company name, surgical pack 180 identifier, manufacture date, lot number, and the like. The surgical pack 180 identifier may identify the type of surgery the pack of surgical instruments 180 is configured for, such as, for example, Oral/Pharyngeal 3, which is configured for oral or pharyngeal surgery and include a size 3 suction tongue blade. Once pack 180 and the surgical instruments are sterilized, the pack 180 and surgical instruments are enclosed in a sealed package (not shown). Optional cover 183 and optional back side 184 may be excluded and the base 182 and surgical instruments, may be sealed in an outer packaging, such as, for example, plastic, that includes the surgical pack 180 identifier.

Base 182 is configured to retain the set of surgical instruments. The set of surgical instruments includes a multi-functional hemostat 110, a multi-functional Allis forceps 120, a ring blade holder 130, a suction tongue blade 140, a palate retractor 150, a multi-function tri-view surgical instrument 160 that includes a mirror, a tissue retractor and spatula retractor or elevator, and a curette 170. Pack 180 may contain other selected sets of surgical instruments depending on the type of surgery for which pack 180 is configured. In addition, the pack of surgical instruments may be custom ordered or customized for a particular surgeon, or surgery center's needs.

Base 182 is configured to hold the surgical instruments in positions that allow for the surgical instruments to be sterilized. Accordingly, a sterilization agent may flow around and/or contact all of the surgical instruments. In addition, base 182 is configured to hold the surgical instruments so that they may be removed in the order that they are needed in a surgery and so that they take up less storage space than conventional trays of surgical instruments.

Use of pack 180 reduces OR time. The surgical instruments are pre-selected for the type of surgery and are organized to be removed from pack 180 in the order that the surgical instruments are needed for that particular surgery. Thus, the typical preparation time that is required for an assistant to unwrap and organize a large set of surgical instruments, many of which are not required for the specific surgery is eliminated.

As discussed in more detail below, base 182 has a number of tabs and retaining members that are made from cut-outs/punch-outs in the base 182. The cut-outs/punch outs are bent upward and configured to retain the surgical instruments.

Base 182 includes retaining member 185A. Retaining member 185A includes interlocking flaps 185, 186. Interlocking flaps 185 and 186 are partially cut-out of base 182 and wrap around the multi-functional hemostat 110 and multi-function Allis 120 and interlock as shown. A pair of tabs 187 are partially cut-out of base 182 and are bent upward and outward. Tabs 187 are configured to fit through the ring handles of Allis forceps 120. Similar, a pair of tabs 188 are partially cut-out of base 182 and are bent upward and outward. Tabs 188 are configured to fit through the ring handles of hemostat 110. At least a portion of hemostat 110 is located on top of Allis forceps 120 and interlocking flaps 185, 186 wrap around hemostat 110 and Allis forceps 120 to retain them in place. Packaging space is saved by allowing earlier used surgical instruments to be placed on or above portions of later uses surgical instruments. In addition, such overlap places the “next up” surgical instrument in a ready position.

Base 182 includes a retention tab 190 that is partially cut-out of base 182. Retention tab 190 has an opening therethrough to receive a handle of the multi-function tri-view surgical instruments 160 to secure the surgical instrument in place. A second tab 191 is partially cut-out of base 182 and is bent upward to retain an upper portion of multi-function tri-view surgical instrument 160. Base 182 includes a retention tab 192. Retention tab 192 is partially cut-out of base 182 and bent upward. Retention tab 192 has an opening that is configured to receive and retain a portion of suction tongue blade 140. A second retention tab 193 is also partially cut-out of base 182 and is bent upward to retain a second portion of suction tongue blade 140. Another tab (not shown) bends upward and fits into the opening (not shown) in the suction tube 178 of the suction blade 140. Base 182 includes a tab 177 bends upward and fits into the channel (not shown) of ring blade holder 130. Base 182 also includes a pair of retention tabs 194, 195. Retention tabs 194, 195 are partially cut-out of base 182 and bent upward. Retention tabs 194, 195 have an opening that is configured to receive and retain a portion of ring blade holder 130. Base 182 includes another pair of retention tabs 196, 197. Retention tabs 196, 197 are partially cut-out of base 182 and bent upward. Retention tabs 196, 197 have an opening that is configured to receive and retain a portion of palate retractor 150. Another retention tab 198 is partially cut-out of base 182 and bent upward. Retention tab 198 has an opening therethrough that is configured to receive a handle of the curette 170. A final retention tab 199 is partially cut-out of base 182 and is bent upward and configured to retain an upper portion of curette 170. In this exemplary pack, retention tabs 191, 192, 194, 195, 196, 197 and 199 have an opening and a channel that extends to the end of the retention tab. The opening may be circular. The surgical instrument is pushed through the channel and seated in the opening. The channel is configured to be positioned over part of the surgical instrument to retain the surgical instrument in place.

This package configuration provides a presorted set of surgical instruments. The package is configured to allow an operator or surgeon to “open and operate.” The package configuration provides a ready to use set of surgical instruments that are preselected for a selected type of surgery. As described in more detail below, the surgical set of instruments may be an expandable set of surgical instruments, and/or an expandable set that includes multi-functional surgical instruments. For example, selected surgical instruments are configured to function as multiple types of instruments, e.g. the forceps may be used as forceps or may be disassembled and used as, for example, one or more retractors. Selected surgical instruments may be disassembled and combined with parts of one or more other disassembled surgical instruments. These combined parts may be referred to herein as hybrid surgical instruments. The hybrid surgical instruments may have jaw portions or different working end portions. The jaw portions or working end portions of the hybrid surgical instruments have different configurations. In addition, selected surgical instruments are multi-functional and perform the functions of multiple prior art surgical instruments. These surgical instruments may also be referred to herein as hybrid surgical instruments or multi-function surgical instruments.

In addition, the package configuration reduces the storage space required for storing the instruments because they are arranged with one or more surgical instruments being located on top of one or more other surgical instruments. In addition, because one or more of the surgical instruments are configured to perform multiple functions, less surgical instruments are required. In addition, because some of the surgical instruments are readily separable, they may be broken down to function as other surgical instruments and/or combined with other readily separable parts to form additional surgical instruments.

Multi-functional tri-view surgical instrument 160 is placed in pack 180. Suction tongue blade 140 is placed in pack 180 and extends over multi-functional tri-view surgical instrument 160. Palate retractor is placed in pack 180 and ring blade holder 130 is place in pack 180. This allows the surgical instruments to be removed from pack 180 in the order that they are needed. Ring blade holder 130 is typically removed first. Suction tongue blade 140 is removed next and secured to ring blade holder 130 to form a mouth gag. The mouth gag is connected to a suction hose (not shown) and placed in use. Hemostat 110 is removed. Palate retractor 150 is removed and placed in use with the hemostat 110. Multi-functional tri-view surgical instrument 160 and curette 170 may then be removed when needed. In addition, the Allis forceps 120 may be removed and placed into use.

The tray 102 of surgical instruments or pack 180 of surgical instruments include at least one surgical instrument that is configured for multiple tasks. In some instances, the tray or pack of surgical instruments includes at least one surgical instrument that may be readily disassembled into separate parts. For example, the tray of surgical instruments may include a hemostat or forceps that may be used as a hemostat or forceps and is readily separated into separate pieces wherein one or more of the two separate pieces may be used individually as a retractor. In some instances, the tray 102 or pack 180 of surgical instruments includes at least two instruments, wherein the first surgical instrument may be readily separated into pieces and the second surgical instrument may be readily separated into pieces and one or more pieces of the first surgical instrument may be combined with one or more pieces of the second surgical instruments to form a hybrid surgical instrument. Thus, the surgical set is expandable.

In some instances, the tray 102 or pack 180 of surgical instruments 100 includes 10 or fewer surgical instruments. In some instances, the tray 102 or pack 180 of surgical instruments 100 includes 9 or fewer surgical instruments. In some instances, the tray 102 or pack 180 of surgical instruments 100 includes 8 or fewer surgical instruments. In some instances, the tray 102 or pack 180 of surgical instruments 100 includes 7 surgical instruments. As used in this paragraph, a multi-functional surgical instrument is counted as 1 surgical instrument. In addition, a surgical instrument that may be disassembled to form a second surgical instrument is counted as 1 surgical instrument. In addition, 2 surgical instruments that are separable and combinable to form a third surgical instrument, are counted as 2 surgical instruments.

The surgical instruments disclosed herein may be single use surgical instruments. The surgical instruments are preferably made from one or more polymers. Single use surgical instruments are preferably made from a non-metallic material. Single-use surgical instruments are preferably not made of stainless steel or surgical steel. The single-use surgical instruments may be devoid of metallic springs. The single-use surgical instruments may be devoid of screws. The single-use surgical instruments may be devoid of small parts. Small pats are parts that would fit down a person's aero digestive track. Small parts smaller than, for example, 10 millimeters. In some instances, the single-use surgical instruments are recyclable. After the surgical procedure is completed, the surgical instruments are simply placed in a container for disposal or recycling. This procedure saves valuable OR time as the surgical instruments do not need to be sprayed down and prepped to begin the sterilization process. In addition, single use surgical instruments do not need to be cleaned, inspected or sterilized after use. Accordingly, there are no costs associated with cleaning, disassembling the mouth gag, reassembling the mouth gag, inspecting and sterilizing the surgical instruments after the surgical procedure.

In some instances, one or more of the surgical instruments, as described herein, may be made of surgical steel or other conventional material and may be reusable. When the surgical instrument is readily disarticulated, it is easier to clean, inspect, repair and sterilize. Because the exemplary surgical instruments do not have small parts or do not need tools to dissemble, the cleaning and inspecting time is also greatly reduced. Furthermore, many of the surgical instruments are configured to perform multiple tasks or functions and thus, the number of surgical instruments required for a particular surgical procedure may be reduced. Thus, even when made of conventional material and reused, the surgical instruments disclosed herein reduce cost and OR time.

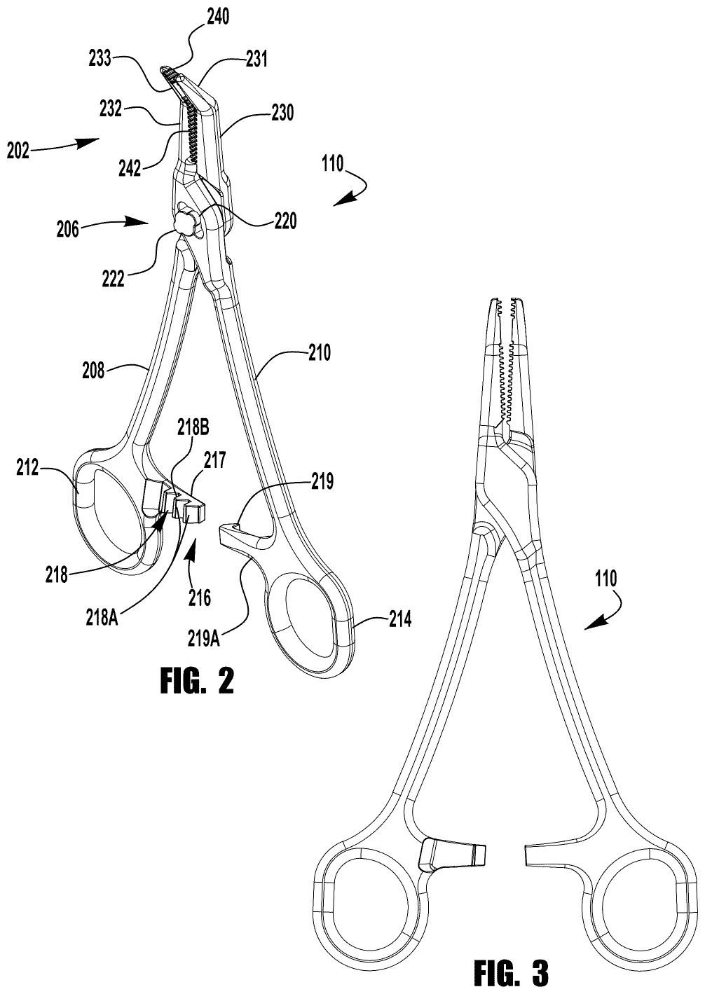

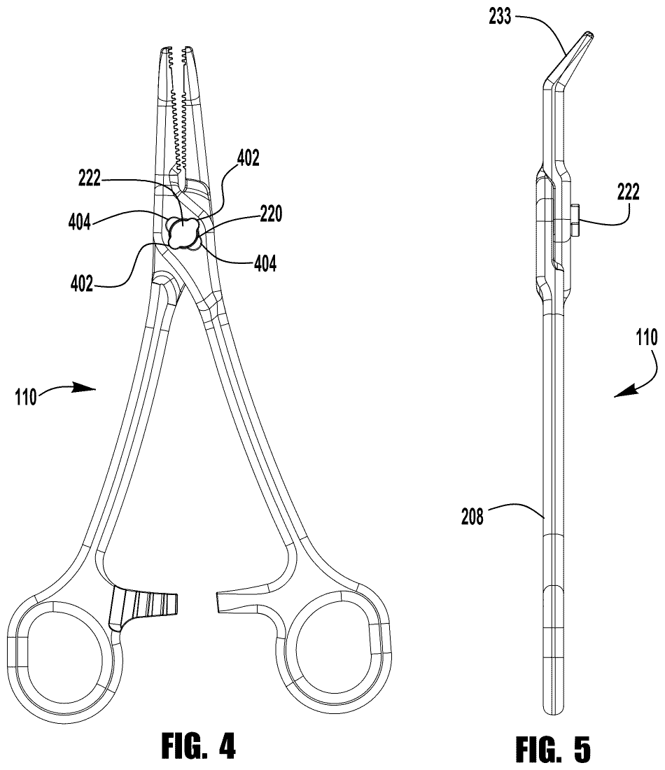

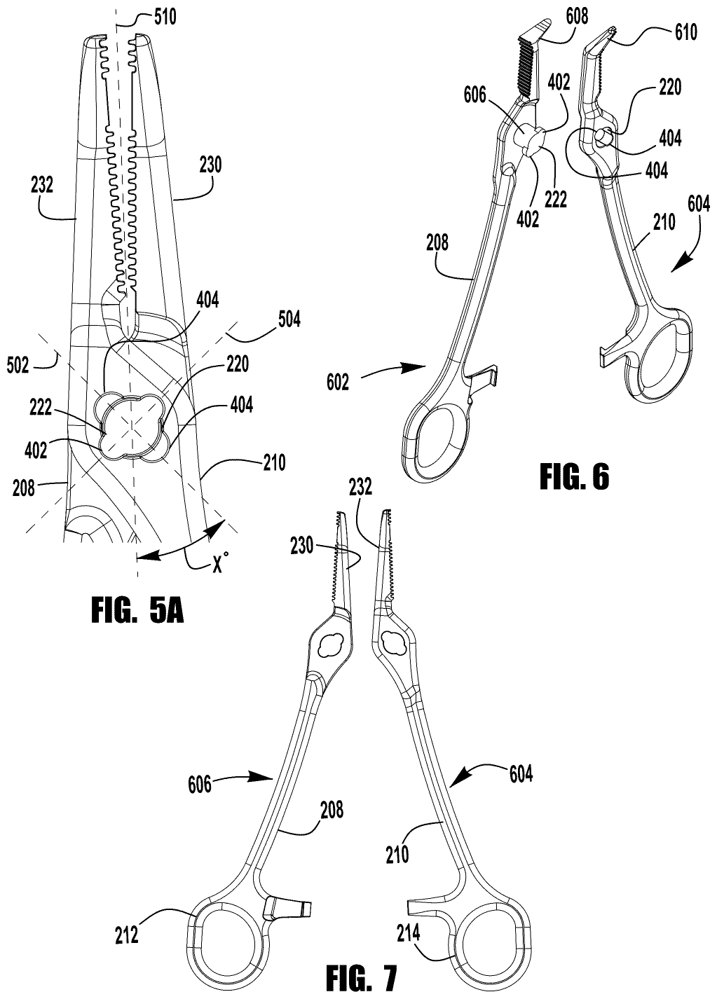

FIGS. 2-8 disclose an exemplary modified hemostat 110. Hemostat 110 includes a first shank 208. First shank 208 has a ring handle 212 located a first end and a jaw 230 located at a second end. Hemostat 110 includes a second shank 210. Second shank 210 has a ring handle 214 located on a first end and a jaw 232 located on a second end. The first shank 208 and the second shank 210 are pivotally, and releasably connected at releasable connector 206 such that as the first ring handle 212 and second ring handle 214 are moved toward each other, the first jaw 230 and the second jaw 232 move toward one another.

Hemostat 110 is made of two individual parts, first shank 208 and second shank 210, that may be readily separated and readily combined. Hemostat 110 does not require a tool to separate shanks 208, 210. Shank 208 and shank 210 are simply rotated to a selected position and separated.

Hemostat 110 does not contain any small parts, such as, for example, rivets, pins, screws, bolts or the like. Accordingly, hemostat 110 does not have small parts that may separate from hemostat 110 and cause injury or increase OR time. Hemostat 110 is devoid of any rivets, screws, bolts, pins. Each shank 208, 210 may be molded as a single piece. In some instances, the shanks are injection molded. In some instances, the shanks 208, 210 are made of one or more polymers. In some instances, the hemostat 110 is single use instrument and is disposed of after a single use.

A ratchet locking mechanism 216 is located proximate the first and second ring handles 212, 214. The ratchet locking mechanism 216 includes a plurality of catches 218 formed by ridges 218A and valleys 218B on extension member 217 that is connected to the first shank 208 and a latch 219 on extension member 219A that extends outward from second shank 210. The plurality of catches 218 allow hemostat 110 to lock the jaws 230, 232 closed. The locked jaws 230, 232 apply varying pressures dependent upon which catch 218 is engaged with latch 219. While three catches 218 are shown in the illustrated hemostat, more or fewer catches 218 may be used.

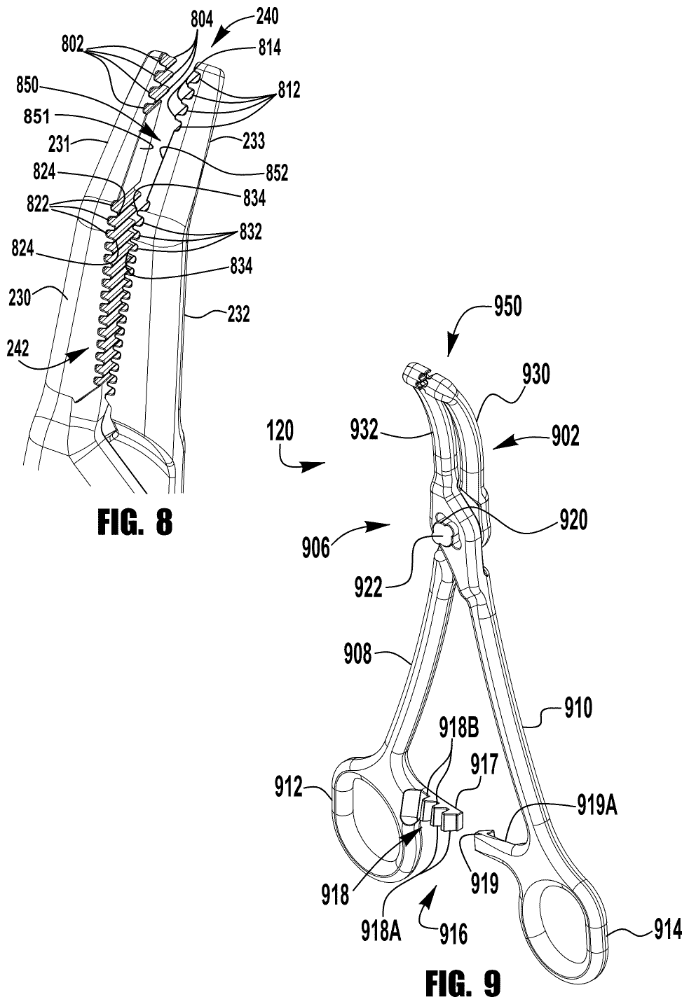

The jaws 230, 232 may be straight, curved or angled (as shown). In this exemplary embodiment, jaw 230 includes an angled outer portion 231. Jaw 232 includes a corresponding angled outer portion 233. Jaws 230, 232 may include optional serrations 240 on respective outer portions 231, 233. Jaws 230, 232 may include optional serrations 242 on their respective inner portions. As shown in more detail in FIG. 8, serrations 240 are located along the angled tip of jaws 230, 232. Serrations 240 include a plurality of ridges 804 and valleys 802 on jaw 230 and a plurality of ridges 814 and valleys 812 on jaw 232. The ridges 804 are configured to mesh with valleys 812 and ridges 814 are configured to mesh with valleys 802. Similarly, optional serrations 242 are located along the inner portion of jaws 230, 232. Serrations 242 include a plurality of ridges 824 and valleys 822 on jaw 230 and a plurality of ridges 834 and valleys 832 on jaw 232. The ridges 824 are configured to mesh with valleys 832 and ridges 834 are configured to mesh with valleys 822.

Jaws 230 and 232 include flat sections 851, 852 respectively. The flat sections 851, 852 are configured to be a needle holder 850 so that multi-functional hemostat 110 functions as a needle driver as well as a hemostat. In this exemplary embodiment, the needle holder 850 is the area along the elongated flat sections 851, 852 that extend along the angled outer portion 231 and 232 of jaws 230, 232.

Shank 208 and shank 210 are releasably coupled at releasable connector 206. Releasable connector 206 allows for the shanks 208 and 210 to be readily connected to one another and readily disconnected from one another. The releasable connector 206 is configured such that a member of a surgical staff can readily disconnect and reconnect the shanks 208, 210.

Connector 206 includes a shaft 606 (FIG. 6). A first end of shaft 606 is connected to shank 208. One or more retaining members 402 are located at the second end 222 of the shaft 606. The retaining members 402 are shown as a first lobe and a second lobe and the first lobe and second lobe are located 180 degrees from one another.

Connector 206 includes an opening 220 in shank 210. At least a portion of opening 220 has a circular cross-sectional shape. The circular cross-sectionally shaped portion is slightly larger than the diameter of the shaft 606. A second portion 404 of opening 220 is configured to be slightly larger than the one or more retaining members 402. When the second portion 404 of opening 220 is aligned with the one or more retaining members 402, the first shank 208 and the second shank 210 may be readily separated from one another by pulling the first shank 208 and the second shank 210 away from each other.

Other shapes of retaining members 402 may be used provided they allow the first and second shanks 208, 210 to smoothly pivot with respect to one another while maintaining the coupling between the first shank 208 and the second shank 210 along a first range of motion and allow for decoupling at a second point in the range of motion.

With respect to FIG. 5A, a centerline 502 extends across opening 220 and second portion 404 of opening 220. A second centerline 504 extends across shaft 606 and retaining members 402. Releasable connector 206 maintains the coupled connection between shanks 208, 210. When centerline 502 lines up with centerline 504, shank 208 and shank 210 may be separated by pulling them away from one another.

FIG. 5A illustrates a third centerline 510 that is substantially perpendicular to at least a portion of jaw 230. In some embodiments, the angle X between center line 510 and centerline 502 must be greater than 90 degrees before centerline 502 and centerline 504 line up so that the fist shank 208 and second shank 210 may be separated. In some embodiments, the angle X between center line 510 and centerline 502 must be greater than 100 degrees before centerline 502 and centerline 504 line up. In some embodiments, the angle X between center line 510 and centerline 502 must be greater than 110 degrees before centerline 502 and centerline 504 line up. In some embodiments, the angle X between center line 510 and centerline 502 must be greater than 120 degrees before centerline 502 and centerline 504 line up. In some embodiments, the angle X between center line 510 and centerline 502 must be greater than 130 degrees before centerline 502 and centerline 504 line up. In some embodiments, the angle X between center line 510 and centerline 502 must be greater than 140 degrees before centerline 502 and centerline 504 line up.

In other embodiments, opening 220 may have a substantially circular cross-section. In such an embodiment, shaft 606 may include one or more retention members (not shown) that when in an unbiased position have an outer diameter that is greater than the diameter of the opening 220 and a biased position that has an outside diameter that is smaller than the diameter of opening 220. In such an embodiment, the retention members are readily deflected radially inward to the biased position. Thus, to readily separate the two shanks from one another, a user simply deflects the retention members (not shown) and pulls them through the opening 220.

When hemostat 110 is disassembled as shown in FIG. 6, shank 208 and hook 608 may be used for other functions, such as, for example, as a retractor, elevator, trocar or other purposes. Similarly, shank 210 and hook 610 may be used as a retractor or other purpose. Thus, hemostat 110 may perform this additional function or task.

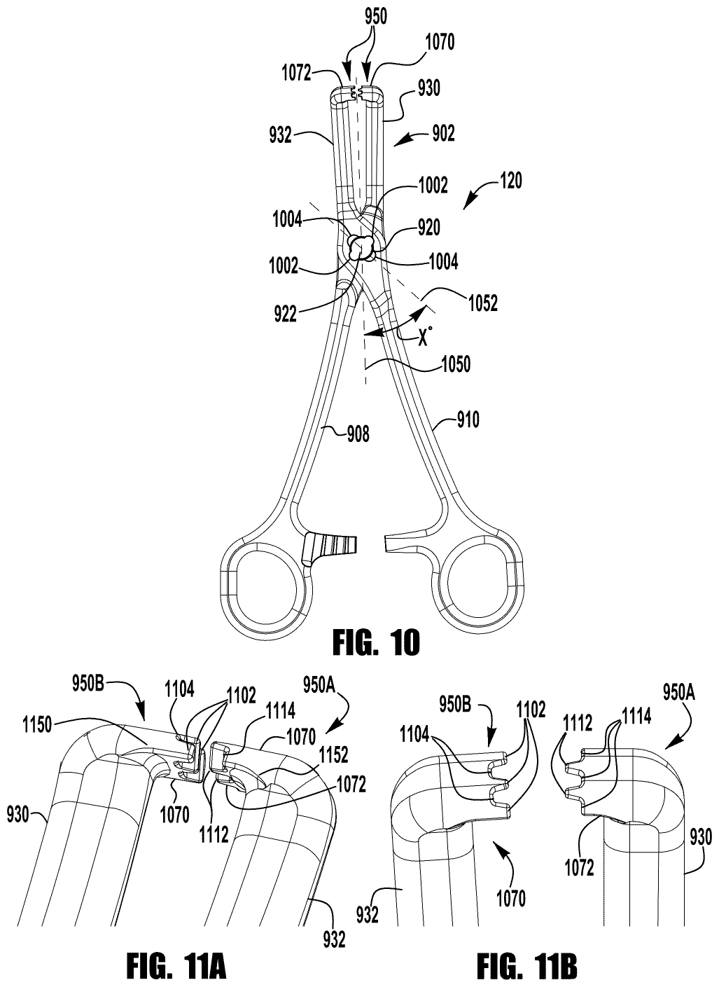

FIGS. 9 through 11B illustrate an exemplary embodiment of a multi-function Allis forceps 120. Allis forceps 120 includes a includes a first shank 908. First shank 908 has a ring handle 912 located a first end and a jaw 930 located a second end. Allis forceps 120 includes a second shank 910. Second shank 910 has a ring handle 914 located on a first end and a jaw 932 located on a second end. The first shank 908 and the second shank 910 are pivotally, and releasably connected at releasable connector 906 such that as the first ring handle 912 and second ring handle 914 are moved toward each other, the first jaw 930 and the second jaw 932 move toward each other.

Allis forceps 120 is made of two individual parts, first shank 908 and second shank 910, that may be readily separated and readily combined. Allis forceps 120 does not contain any small parts, such as, for example, rivets, screws, pins, or the like. Accordingly, allis forceps 120 does not have small parts that may separate from hemostat 110 and cause injury or increase OR time. Allis forceps 120 is devoid of any rivets, screws, or pins. Each shank 908, 910 may be molded as a single piece. In some instances, the shanks are injection molded. In some instances, Allis forceps 120 are made of one or more polymers. In some instances, Allis forceps 120 are single use surgical instruments are configured to be used only a single time and then disposed of and/or recycled. In some instances, Allis forceps 120 are made of a non-metallic material.

A ratchet locking mechanism 916 is located proximate the first and second ring handles 912, 914. The ratchet locking mechanism 916 includes a plurality of catches 918 formed by ridges 918A and valleys 918B on extension member 917 that is connected to the first shank 908 and a latch 919 on extension member 919A that extends outward from second shank 910. The plurality of catches 918 allow a surgeon to lock the jaws 930, 932 closed. The locked jaws 930, 932 apply varying pressures dependent upon which catch 918 is engaged with latch 919.

The jaws 930, 932 may be straight, angled, or curved as shown. Located at the end of jaw 930 is an inwardly extending projection 1150 (FIGS. 11A, 11B) that forms a hook 1070. The inwardly extending projection 1150 extends inward from the jaw 930 far enough so that hook 1070 may be used as a retractor during surgery. Similarly, located at the end of jaw 932 is an inwardly extending projection 1152 that forms a hook 1072. The inwardly extending projection 1152 extends inward from the jaw 932 far enough so that hook 1072 may be used as a retractor during surgery.

In some instances, inwardly extending projections 1150, 1152 extend at least 3 millimeters (“mm”) from the edge of the jaw. In some instances, inwardly extending projections 1150, 1152 extend at least 4 millimeters (“mm”). In some instances, inwardly extending projections 1150, 1152 extend at least 5 millimeters (“mm”). In some instances, inwardly extending projections 1150, 1152 extend at least 6 millimeters (“mm”). In some instances, inwardly extending projections 1150, 1152 extend at least 7 millimeters (“mm”). In some instances, inwardly extending projections 1150, 1152 extend at least 8 millimeters (“mm”). In some instances, inwardly extending projections 1150, 1152 extend at least 9 millimeters (“mm”). In some instances, inwardly extending projections 1150, 1152 extend at least 10 millimeters (“mm”).

Hook 1070 extends along a hook axis and jaw 930 extends along a jaw axis. Similarly, Hook 1072 extends along a hook axis and jaw 932 extends along a jaw axis. In some instances, the angle between the hook axis and the jaw axis is between 40° and 140°. In some instances, the angle between the hook axis and the jaw axis is between 50° and 130°. In some instances, the angle between the hook axis and the jaw axis is between 60° and 120°. In some instances, the angle between the hook axis and the jaw axis is between 70° and 110°. In some instances, the angle between the hook axis and the jaw axis is between 80° and 100°. In some instances, the angle between the hook axis and the jaw axis is between 85° and 95°. In some instances, the angle between the hook axis and the jaw axis is between 87° and 93°. In some instances, the angle between the hook axis and the jaw axis is between 88° and 89°. In some instances, the angle between the hook axis and the jaw axis is 90°. In some instances, the angle between the hook axis and the jaw axis is greater than 90°.

In addition, Allis forceps 120 includes optional engagement members 950 at an end of the inwardly extending projection. Engagement members 950 may be used to, for example, grip tissue. Portion 950A of engagement members 950 include a plurality of ridges 1112 and valleys 1114. Portion 950B of engagement members 950 include a plurality of ridges 1102 and valleys 1004. Ridges 1102 are configured to fit into valleys 1114 and ridges 1112 are configured to fit into valleys 1104.

Shank 908 and shank 910 are releasably coupled at releasable connector 906. Releasable connector 906 allows for the shanks 908 and 910 to be readily connected to one another and readily disconnected to one another. The releasable connector 906 is configured such that a member of a surgical staff can readily disconnect and reconnect the shanks 908, 910. The term releasable connector does not include permanent connectors that cannot be easily connected and disconnected. Examples of permanent connectors are rivets, screws, bolt/nuts and the like.

Connector 906 includes a shaft (not shown) similar to shaft 606 (FIG. 6). Shaft (not shown) is connected to shank 908 on a first end. One or more retaining members 1002 are located at the outer end of the shaft (not shown). The retaining members 1002 are shown as a first lobe and a second lobe located 180 degrees from one another.

Connector 906 includes an opening 920 in shank 910 to receive shaft 606. At least a portion of opening 920 has a circular cross-sectional shape. The circular cross-sectionally shaped portion is slightly bigger than the diameter of the shaft (not shown). A second portion 1004 of opening 920 is configured to be slightly larger than the one or more retaining members 1002. When the second portion 1004 of opening 920 is aligned with the one or more retaining members 1002, the first shank 908 and the second shank 910 may be readily separated from one another.

Other shapes of retaining members 1002 may be used provided they allow the first and second shanks 908, 910 to pivot with respect to one another while maintaining the coupling between the first shank 908 and the second shank 910 along a first range of motion and allow for decoupling at a second point in the range of motion.

A centerline (not shown) extends across opening 920 and second portion 1004 of opening 920. A second centerline (not shown) extends across shaft outer surface 922 and retaining members 1002. Releasable connector 906 maintains the coupled connection between shanks 908, 910 until the centerlines line up and then shank 908 and shank 910 may be separated by pulling them away from one another.

Shank 910 is rotated with respect to shank 908 to align the retaining members 1002 and the portion 1004 of the opening 920. A first centerline 1050 extends through hemostat 120. A second centerline 1052 extends through and bisects opening 220 and portions 1004 of opening 920. An angle X may be measured between the two centerlines, wherein shank 910 must be rotated X degrees to cause the retaining members to align with their respective openings. In some embodiments, the angle X between center line 1050 and centerline 1052 must be greater than 90 degrees before the retaining members 1002 and portions 1004 of opening 920 line up. In some embodiments, the angle X between center line 1050 and centerline 1052 must be greater than 100 degrees before the retaining members 1002 and portions 1004 of opening 920 line up. In some embodiments, the angle X between center line 1050 and centerline 1052 must be greater than 120 degrees before the retaining members 1002 and portions 1004 of opening 920 line up. In some embodiments, the angle X between center line 1050 and centerline 1052 must be greater than 130 degrees before the retaining members 1002 and portions 1004 of opening 920 line up. In some embodiments, the angle X between center line 1050 and centerline 1052 must be greater than 140 degrees before the retaining members 1002 and portions 1004 of opening 920 line up.

Optionally opening 920 may have a substantially circular cross-section. In such an embodiment, shaft (not shown) may include retention members (not shown) that in an unbiased position have an outer diameter that is greater than the diameter of the opening 920 and a biased position that has an outside diameter that is smaller than the diameter of opening 920. In such an embodiment, the retention members are readily deflected radially inward to the biased position.

When Allis forceps 120 is disassembled, shank 908 and hook 1070 may be used as a retractor. Similarly, shank 910 and hook 1072 may be used as a retractor, or other device.

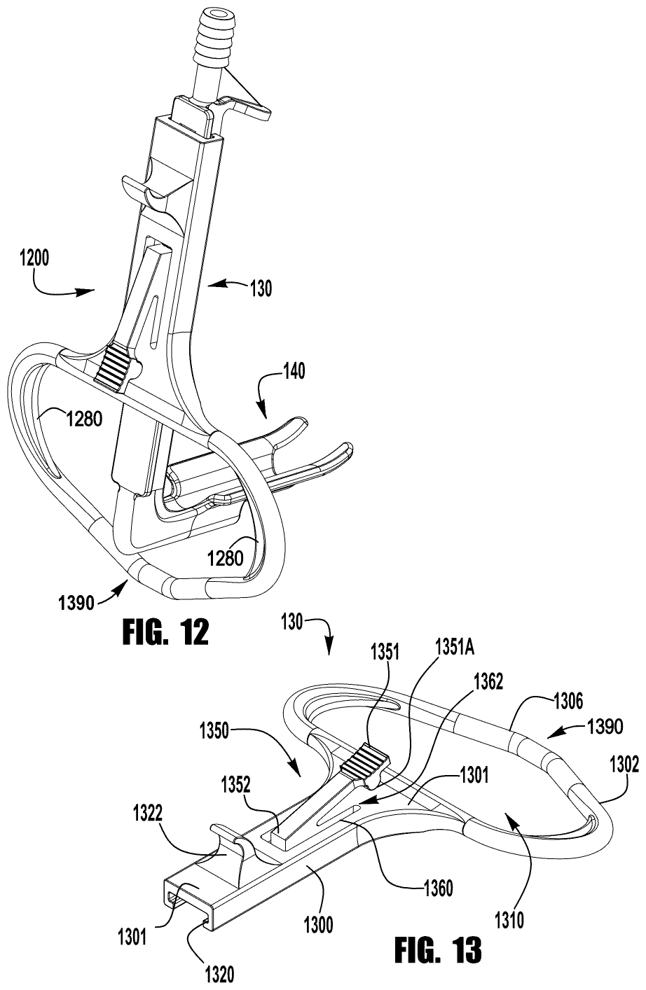

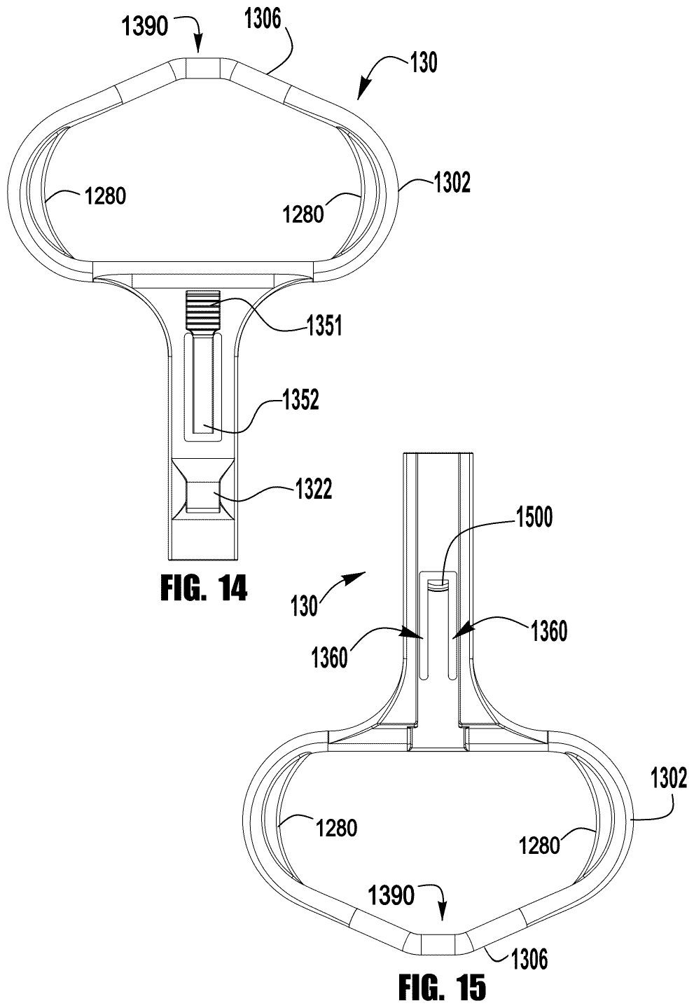

FIG. 12 is a prospective view of a mouth gag 1200. Mouth gag 1200 includes a ring blade holder 130 connected to a suction tongue blade 140. Suction tongue blade 140 and ring blade holder 130 are single use devices. They are made out of one or more polymeric materials. Ring blade holder 130 is a single piece of molded material. Ring blade holder 130 does not contain any small parts, such as, for example, springs, rivets, pins or connectors, that may separate from ring blade holder 130 and cause injury or increase OR time. Ring blade holder 130 is devoid of small separate parts, such as, for example, springs, screws, rivets and connectors. In addition, ring blade holder 130 does not require any protective covering, such as a silicon sleeve (that is commonly used on surgical steel ring blade holders) to protect a person's teeth from damage during surgery. Ring blade holder 130 includes a body 1300 (FIG. 13). Body 1300 has a ring member 1302. Ring member 1302 forms a working opening 1310. Upper portion 1306 of ring member 1302 contacts the roof of a patient's mouth and/or maxillary dentition (i.e. teeth of upper jaw) when the mouth gag 130 is used during a procedure. Ring member 1302 includes one or more optional buttresses 1280. Two buttresses 1280 are shown in the exemplary embodiment. Buttresses 1280 may provide additional strength to ring member 1302. In addition, buttresses 1280 may be configured for attachment of auxiliary devices. Such buttress configurations may include one or more flat portions. Buttresses 1280 may have various lengths and shapes and the one or more buttresses 1280may be located along one or more areas around the perimeter of the ring member 1302. Buttresses 1280, along with the ring member 1302, may be configured to provide for the placement of clips (not shown) or rings (not shown) that could be used to attach additional and/or auxiliary devices, such as, for example, lip and cheek retractors. The buttresses 1280 and ring member 1302 may allow the mouth gag 1200 to function similarly to a Dingman device (not shown). The buttresses 1280 may be configured for attaching fiber optic cables (not shown) or other devices (not shown). The attachments (not shown) may be fixed or adjustable. Ring member 1302 is configured with various curves and angles to conform with facial and oral anatomy and to allow for attachment of the pallet retractor. Ring member 1302 is configured for attachment of pallet retractor 150. Pallet retractor 150 engages ring member 1302 along portion 1390. The configuration of 1390 is a flat or horizontal section that matches the body of the palate retractor 150. The width of the pallet retractor connection member 1910 may be substantially the same as the width of portion 1390 of ring member 1302. This may increase the stability of palate retractor 150, and also facilitate in properly locating palate retractor 150. Body 1300 includes a channel 1320 for receiving a spine or shaft of suction tongue blade 140. Body 1300 includes a handle 1322 located on upper surface 1301.

Body 1300 includes a latching mechanism 1350. Latching mechanism 1350 includes a release member 1351. Release member 1351 optionally includes a plurality of ridges or knurls. An optional stop member 1351A is located below release member 1351 which prevents overtravel of the latching mechanism 1350. A channel 1360 through surface 1310 extends around three sides latching mechanism 1350 forming a flexible resilient hinge area 1362. Resilient hinge area 1362 provides a biasing force that when deflected from its resting position, biases latching mechanism 1350 toward its latching position.

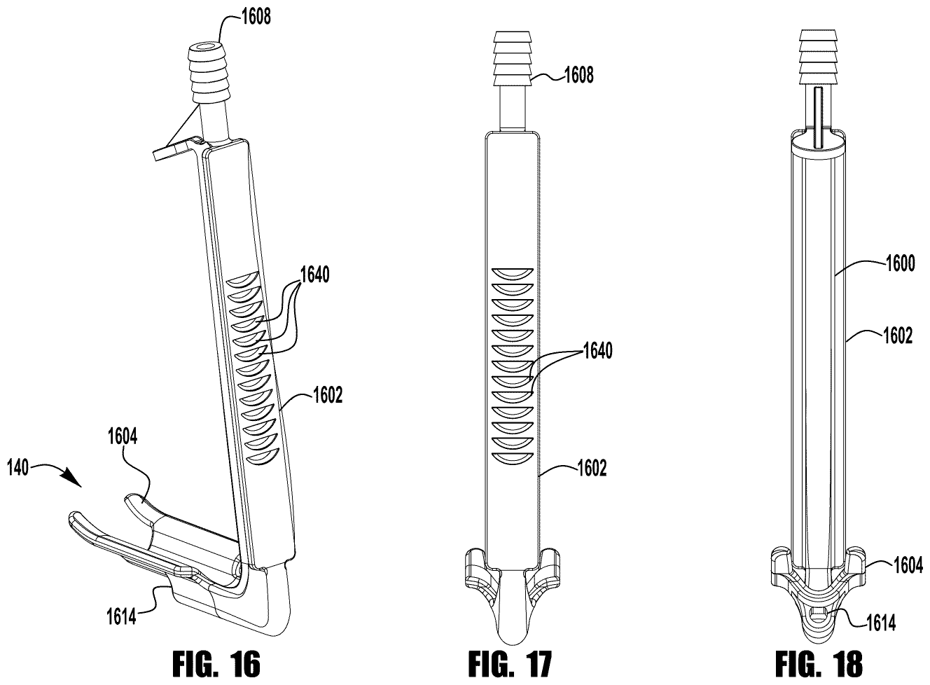

Latching mechanism 1350 has an end 1352 of opposite hinge area 1362. Located on the bottom of end 1352 is a latch 1500 (FIG. 15). Latch 1500 engages with one of the catches 1640 (FIG. 16) on the spine of suction tongue blade 140 when the ring blade holder 130 is connected to suction tongue blade 140. The latch 1500 extends down low enough so that when the spine 1602 of suction tongue blade 140 is inserted in channel 1320, upward pressure is placed on latch 1500 creating deflection in resilient hinge area 1362 biasing the latch to fully engage catch 1640. To release ring blade holder 130 from tongue blade 140, release member 1351 is pushed toward surface 1301, which causes end 1352 and latch 1500 to deflect away from the catch 1640 of spine 1602 of tongue blade 140.

FIGS. 16-18 are an exemplary embodiment of a tongue blade 140. Tongue blade 140 includes spine 1602. Spine 1602 is configured to be received by channel 1320 of mouth gag 130. Tongue blade 140 has an engagement surface 1604 that engages the tongue of a patient during surgical procedures. A suction tube connector 1608 is configured to be connected to a suction hose. The body 1610 includes a hollow portion that extends from the suction tube connector 1608 to a suction inlet 1614. Engagement surface 1604 is typically sized according to the size of the patient and comes in varying sizes, see, for example, tongue blades 140, 140A and 140B (FIG. 1). An exemplary tongue blade is shown in U.S. Pat. No. 8,852,169, titled Suction Device for Evacuating Fumes, which is incorporated herein by reference in its entirety.

Suction inlet 1614 is located midline of the tongue blade 140, or along the center of the engagement surface 1604. In some instances, the hollow portion of the body, the suction tube connector 1614, and the suction inlet 1614 are all located along a center line of the tongue blade 140. In some embodiments, at least the suction inlet 1614 is located along a centerline of the engagement surface 1604. In some embodiments, there are at least one suction inlet. In some embodiments, there are at least two suction inlets (not shown). In such embodiments, preferably the at least two suction inlets are located symmetrically about a centerline of the engagement surface 1614. In some embodiments, the at least two suction inlets (not shown) are located along the centerline of the engagement surface 1614. In some embodiments, at least one suction inlet is located proximate a distal end of the engagement surface 1614. In some embodiments, at least one suction inlet is located proximate a proximal end of the engagement surface 1614. In some embodiments, a first suction inlet (not shown) is located on a first side of the engagement surface 1614 and a second suction inlet (not shown) is located on a second side of the engagement surface 1614. In some embodiments, as shown, the suction inlet is at a distal end of the hollow portion of the tongue blade 140. In some embodiments, there are three suction inlets (not shown), one suction inlet located at the end of the hollow portion (1614 as shown), a second suction inlet is located at an angle offset from the fist suction inlet, and a third suction inlet is located at an angle offset from the first suction inlet, and opposite from the second suction inlet. In some embodiments, the offset angle is about 90 degrees.

The suction inlet 1614 evacuates smoke and fumes. The midline, or centerline suction inlet allows the suction inlet 1614 to be located close to the source smoke or fumes irrespective of whether the source is on one side of the blade 1604 or on the other side. In some embodiments, the midline location of the suction inlet 1614 ensures that the suction inlet 1614 is located within 3 inches of the source of the smoke or fumes. In some embodiments, the midline location of the suction inlet 1614 ensures that the suction inlet 1614 is located within 2 ½ inches of the source of the smoke or fumes. In some embodiments, the midline location of the suction inlet 1614 ensures that the suction inlet 1614 is located within 2 inches of the source of the smoke or fumes.

In some embodiments, the tongue blade 140 is configured such that at least one suction inlet is located within 3 inches of the source of the smoke or fumes. In some embodiments, the tongue blade 140 is configured such that at least one suction inlet is location of the suction inlet 1614 ensures that the suction inlet 1614 is located within 2 ½ inches of the source of the smoke or fumes. In some embodiments, the tongue blade 140 is configured such that at least one suction inlet is location of the suction inlet 1614 ensures that the suction inlet 1614 is located within 2 inches of the source of the smoke or fumes.

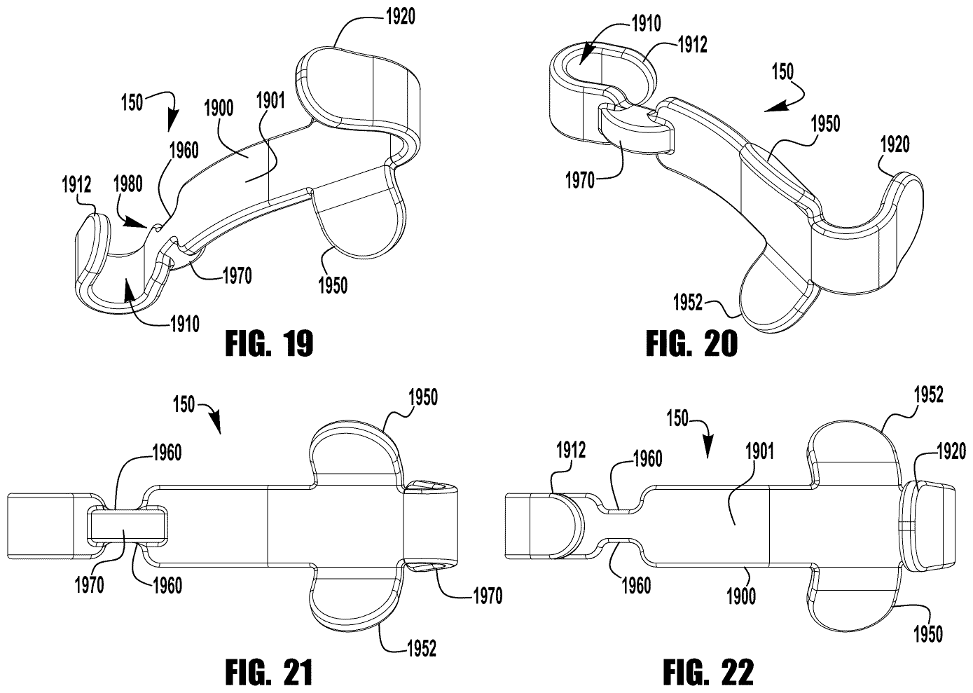

FIGS. 19-22B illustrate an exemplary embodiment of a palate retractor 150. Prior to the innovative palate retractor shown and described herein, surgeons will typically thread a rubber tubing through each of a patient's nasal passageways and back through the mouth and secure the rubber tubing to hold the palate/tissue out of the way. This method of retracting the palate/tissue is not optimal and can possibly displace tissue and obstruct the surgeon's view. It is also time consuming, which increases OR time and may cause irritation or bleeding in the nasal passages of the patient.

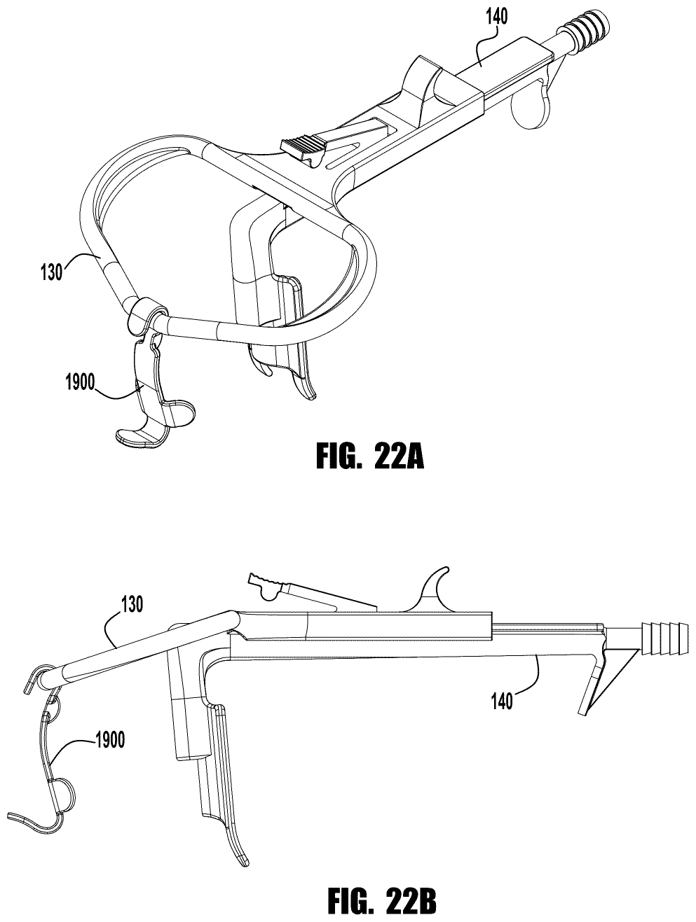

Palate retractor 150 includes a body 1900. The palate retractor is anatomically contoured to a human palate. Body 1900 has a slightly curved shape. The slightly curved shape places the upper surface 1901 of body 1900 proximate the roof a patient's mouth and provides a much better/clearer view for the surgeon and provides better options to grasp and remove tissue. As used with the description of the palate retractor 150 the term “upward” refers to portions that would be toward the roof of a patient's mouth. For example, in FIG. 22, the term upward, or upper, would refer to the direction extending up from the page, e.g. surface 1901 is an upper surface, or upward surface of body 1900. Optionally, body 1900 may be straight.

Located at a first end of body 1900 is mouth gag connection member 1910 formed by curved, or hooked member 1912. As discussed above, palate connection member 1910 is configured to match the portion 1390 of the ring member 1302. Hooked member 1912 is curved upward with respect to surface 1910 of body 1900. Hooked member 1912 is configured to, for example, couple with upper portion 1390 of ring blade holder 130 as shown in FIGS. 22A and 22B, if the mouth gag includes a ring blade holder.

Body 1900 includes a grip member 1980. Grip member 1980 includes indentations 1960 and an arcuate rib 1970. During use, grip member 1980 is configured to be gripped by a surgical instrument, such as, for example, hemostat 110, so that palate retractor 1900 may be easily manipulated during a surgical procedure. In some instances, arcuate rib 1970 may also increase the rigidity of the body 1900 proximate the ring blade holder connection member 1910.

Located on a second end of body 1900 is a palate engagement member 1920. Palate engagement member 1920 is curved upward with respect to surface 1901. Palate engagement member 1920 is curved slightly inward toward the center of body 1900. Palate engagement member 1920 is configured to wrap around the soft palate behind the uvula and elevate the palate when palate retractor is connected to ring blade holder 130 and placed in use during surgery.

Located on body 1900 proximate the palate engagement member 1920 is a pair of wings 1950, 1952. Wings 1950, 1952 are curved downward and outward with respect to surface 1901. The wings 1950, 1952 are configured to engage and hold back tonsils/soft tissue laterally during surgical procedures.

In some instances, palate retractor 150 is made of one or more polymers. In some instances, palate retractor 150 is made of a non-metallic material. In some instances, palate retractor 150 is a single use device and is disposed of after a single use.

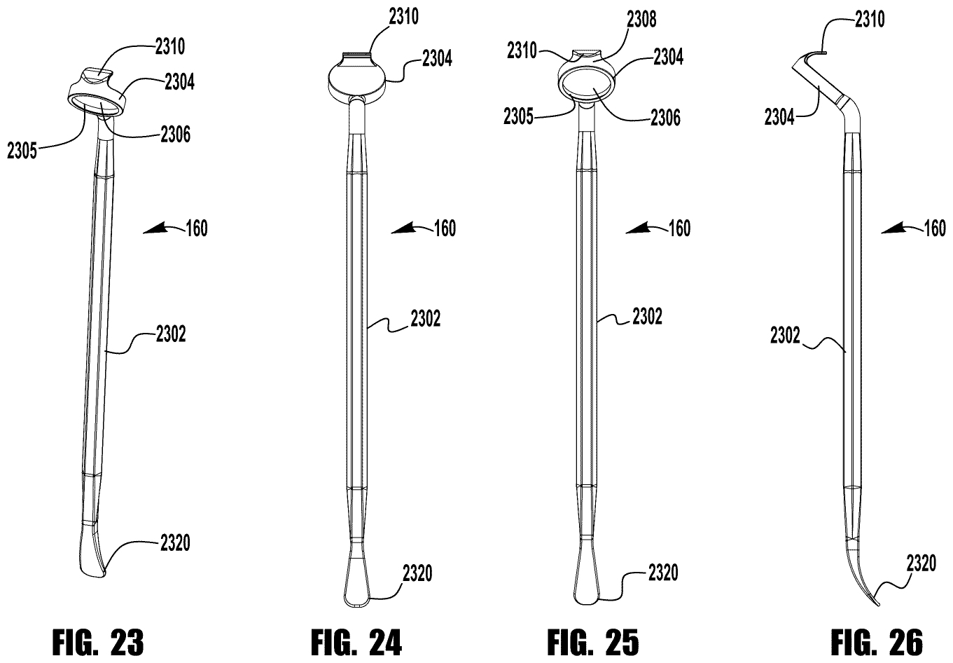

FIGS. 23 through 26 illustrate another exemplary multifunction tri-view surgical instrument 160. Multi-function tri-view surgical instrument 160 has several components that perform three different functions. Multifunction surgical instrument 160 has a handle 2302. Located on a first end of the handle is a mirror head 2304. Mirror head 2304 extends at an angle from handle 2302. The angle can be various angles. Head 2304 includes an annular recess 2305. A mirror 2306 is secured within annular recess 2305. A curved hook 2310 is located on the back side of the angled mirror head 2304. The curved hook 2310 may be used as a retractor to retract or elevate tissue to enhance view of the surgical field. A spatula retractor/elevator 2320 is located on a second end of handle 2302.

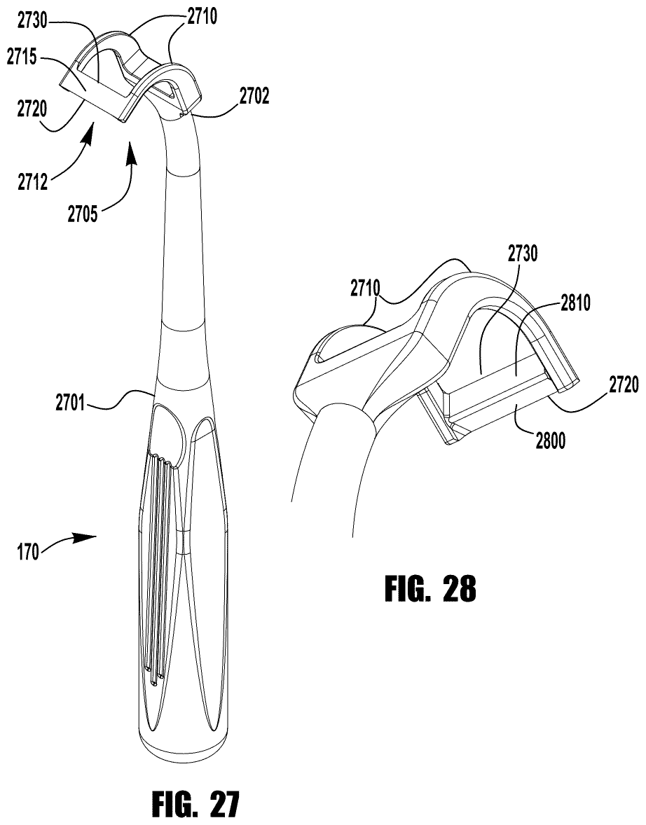

FIGS. 27 and 28 illustrate an exemplary embodiment of a curette 170. Curette 17 has a handle 2701. On a first end 2702 of handle 2701 is a head 2705. Head 2705 incudes a pair of forks 2710. Forks 2710 may be curves, angled or straight. Located at the outer end of the forks is a blade 2712. Blade 2712 has a flat surface 2715, first cutting edge 2720 and a second cutting edge 2730. First cutting edge 2720 and second cutting edge 2730 are both sharp. This allows this curette 170 to be used in both a pulling direction and a pushing direction. Prior art curettes are configured to be used in either a pulling direction or a pushing direction, but not both. A first inclined surface 2800 extends from the first cutting edge. A second inclined surface 2810 extends upward from the second cutting edge 2730. The first inclined surface 2800 and the second inclined surface 2810 may intersect. In some instances, the intersection has a chamfered edge. While blade 2712 is shown as a straight blade, blade 2712 may be curved or angled. In addition, first cutting edge 2720 may have the same shape as second cutting edge 2730 or have a different shape. For example, first cutting edge 2712 may be curved or angled in a first direction and second cutting edge 3730 may be straight, curved or angled in a second direction. In some instances, the second direction is substantially opposite to the second direction.

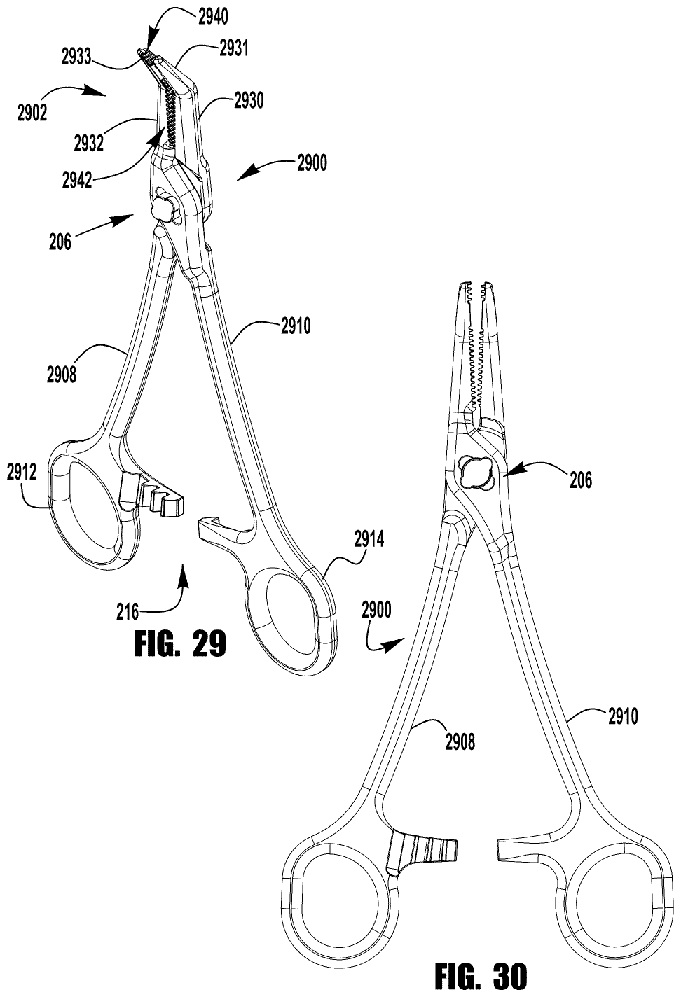

FIGS. 29-34 disclose an exemplary hemostat 2900. Hemostat 2900 includes a first shank 2908. First shank 2908 has a ring handle 2912 located a first end and a jaw 2930 located at a second end. Hemostat 2900 includes a second shank 2910. Second shank 2910 has a ring handle 2914 located on a first end and a jaw 2932 located on a second end. The first shank 2908 and the second shank 2910 are pivotally, and releasably connected at releasable connector 206 such that as the first ring handle 2912 and second ring handle 2914 are moved toward each other, the first jaw 2930 and the second jaw 2932 move toward one another.

Releasable connector 206 is described in detail above and is not redescribed herein.

Hemostat 2900 is made of two individual parts, first shank 2908 and second shank 2910, that may be readily separated and readily combined. Hemostat 2900 does not contain any small parts, such as, for example, rivets or pins. Accordingly, hemostat 2900 does not have small parts that may separate from hemostat 2900 and cause injury or increase OR time. Hemostat 2900 is devoid of any rivets, screws, pins. Each shank 2908, 2910 may be molded as a single piece. In some instances, the shanks are injection molded. In some instances, the shanks 2908, 2910 are made of one or more polymers. In some instances, the hemostat 2900 is single use instrument and is disposed of after a single use. In some instances the hemostat 2900 is made of a non-metallic material.

A ratchet locking mechanism 216 also included. Ratchet locking mechanism 216 is described above in detail and is not redescribed herein.

The jaws 2930, 2932 may be straight, curved or angled (as shown). In this exemplary embodiment, jaw 2930 includes an angled outer portion 2931 and jaw 2932 includes a corresponding angled outer portion 2933. Jaws 2930, 2932 may include optional serrations 2940 on outer portions 2931, 2933. Jaws 2930, 2932 may include optional serrations 2942 on their inner portions.

Jaws 2930 and 2932 include flat sections 3450, 3452 respectively. The flat sections 3450, 3452 are configured to be a needle holder 3460 so that multi-functional hemostat 2900 functions as a needle driver as well. In this exemplary embodiment, the needle holder 3600 is the area along the elongated flat sections 3450, 3452 that extend along both a part of straight portion of jaws 2930, 2932 and a part of the angled outer portion 2931 and 2933 of jaws 2930, 2932. Flat portions 3450A and flat portions 3452A extend along a portion of the angled outer portion 2931 and 2932 and flat portions 3450B, 3452B extend along a portion of the straight portion of jaws 2930, 2932. In some instances, flat portions 3450A and 3450B meet at a vertex of an angle and flat portions 3452A and 3452B meet at a vertex of an angle and a needle may be gripped between jaw 2930 and jaw 2932 between the two vertices.

Shank 2908 and shank 2910 are releasably coupled at releasable connector 206. Releasable connector 206 allows for the shanks 2908 and 2910 to be readily connected to one another and readily disconnected from one another. The releasable connector 2906 is configured such that a member of a surgical staff can readily disconnect and reconnect the shanks 2908, 2910. The term releasable connector does not include permanent connectors that cannot be easily connected and disconnected. Examples of permanent connectors are rivets, screws (that require a screwdriver to remove), bolts/nuts, and the like.

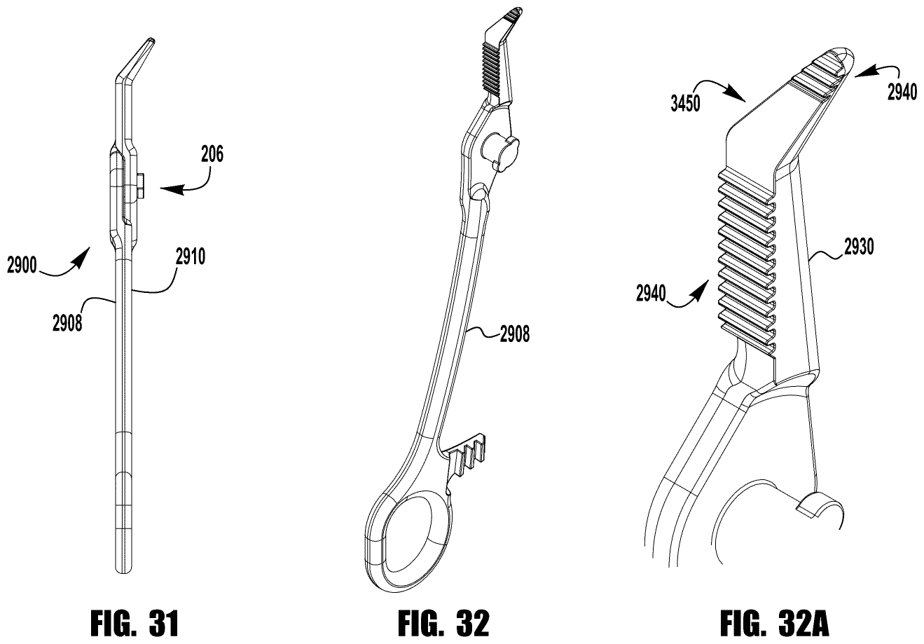

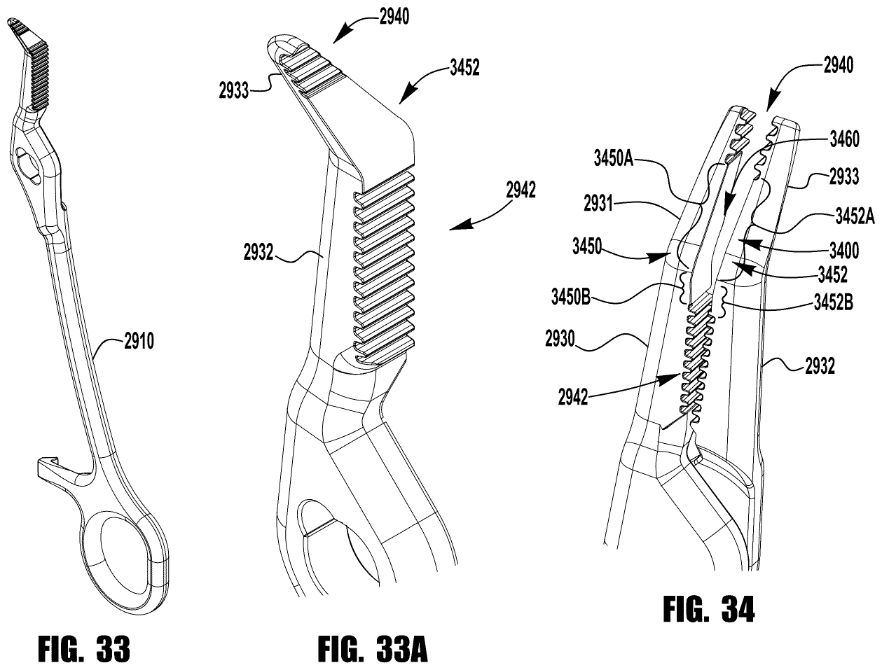

When hemostat 2900 is disassembled as shown in FIGS. 32, 33, shank 2908 angled portion 2931 of jaw 2930 be used as a retractor, a trocar, and/or other purpose. Similarly, shank 2910 and angled portion 2933 may be used as a retractor, a trocar, and/or other purpose. Thus, hemostat 2900 may perform this additional function or task.

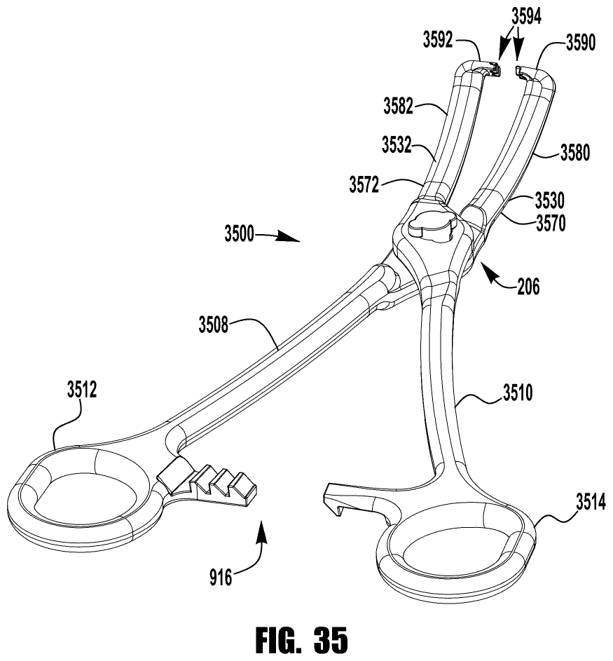

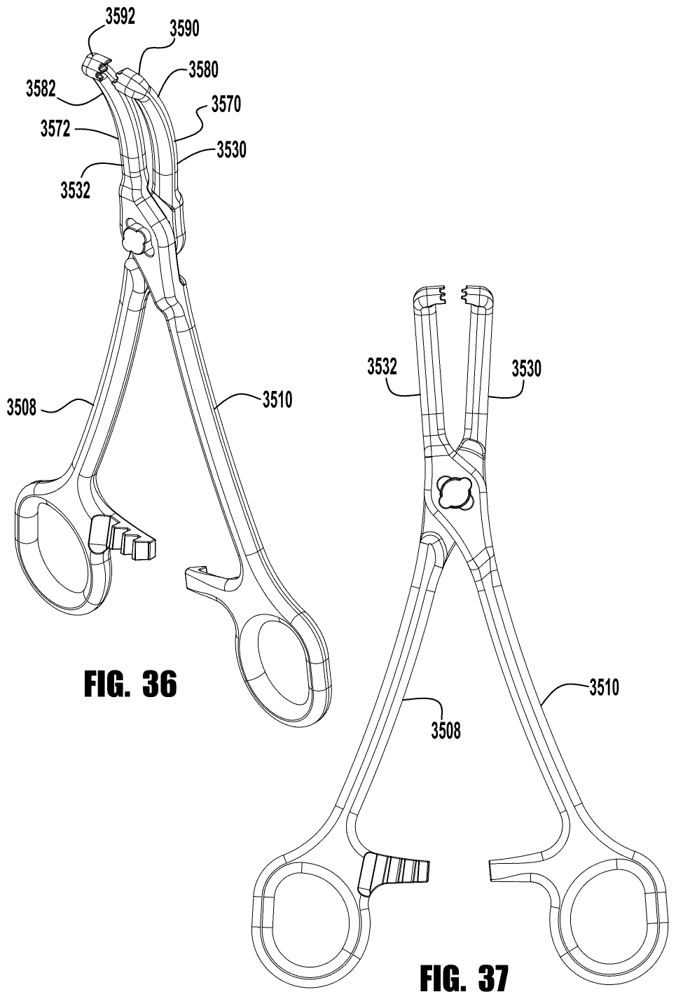

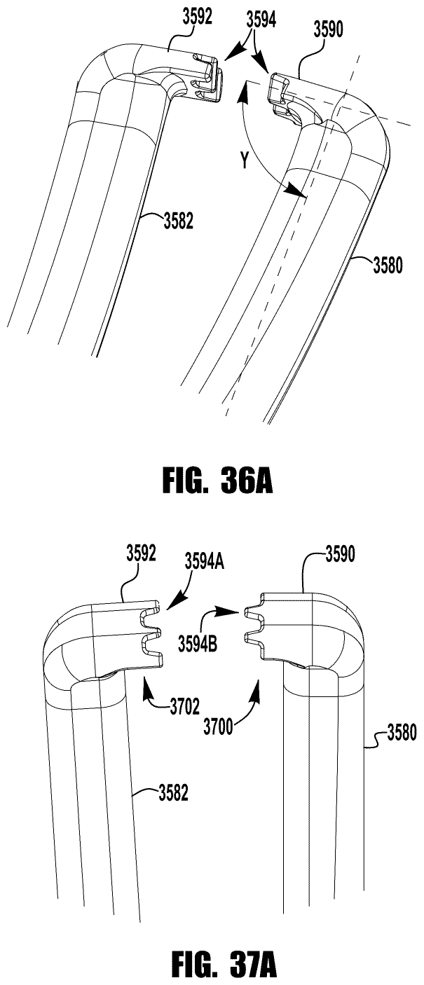

FIGS. 35 through 37A illustrate an exemplary embodiment of a multi-function Allis forceps 3500. Allis forceps 3500 includes a includes a first shank 3508. First shank 3508 has a ring handle 3512 located a first end and a jaw 3530 located a second end. Allis forceps 3500 includes a second shank 3510. Second shank 3510 has a ring handle 3514 located on a first end and a jaw 3532 located on a second end. The first shank 3508 and the second shank 3510 are pivotally, and releasably connected at releasable connector 206 such that as the first ring handle 3512 and second ring handle 3514 are moved toward each other, the first jaw 3530 and the second jaw 3532 move toward each other.

Jaw portion 3530 includes a straight portion 3570 and an angled portion 3580. Similarly, jaw portion 3532 includes a straight portion 3572 and angled portion 3582. Having a portion of jaws 3530, 3532 angled, rather than curved or straight, provides less obstruction of the view for the surgeon and/or provide more options to grasp and remove tissue.

Allis forceps 3500 is made of two individual parts, first shank 3508 and second shank 3510, that may be readily separated and readily combined. Allis forceps 35000 does not contain any small parts, such as, for example, rivets or pins. Accordingly, Allis forceps 3500 does not have small parts that may separate from forceps 3500 and cause injury or increase OR time. Allis forceps 120 is devoid of any rivets, screws, pins. Each shank 908, 910 may be molded as a single piece. In some instances, the shanks are injection molded. In some instances, Allis forceps 3500 are made of one or more polymers. In some instances, Allis forceps 3500 are single use surgical instruments are configured to be used only a single time and then disposed of and/or recycled. In some instances, the Allis forceps 3500 are made of non-metallic materials.

A ratchet locking mechanism 916 is included and is the same as ratchet locking mechanisms described above and is not redescribed herein.

Located at the end of jaw 3530 is an inwardly extending projection 3590 that forms a hook 3700. The inwardly extending projection 3700 extends inward from the jaw 3580 far enough so that hook 3700 may be used as a retractor and more easily facilitate the grasping of tissue during surgery. Similarly, located at the end of jaw 3582 is an inwardly extending projection 3592 that forms a hook 3702. The inwardly extending projection 3592 extends inward from the jaw 3532 far enough so that hook 3702 may be used as a retractor and more easily facilitate the grasping of tissue during surgery.

In some instances, inwardly extending projections 3700, 3702 extend at least 3 millimeters (“mm”) from the edge of the jaw. In some instances, inwardly extending projections 3700, 3702 extend at least 4 millimeters (“mm”). In some instances, inwardly extending projections 3700, 3702 extend at least 5 millimeters (“mm”). In some instances, inwardly extending projections 3700, 3702 extend at least 6 millimeters (“mm”). In some instances, inwardly extending projections 3700, 3702 extend at least 7 millimeters (“mm”). In some instances, inwardly extending projections 3700, 3702 extend at least 8 millimeters (“mm”). In some instances, inwardly extending projections 3700, 3702 extend at least 9 millimeters (“mm”). In some instances, inwardly extending projections 3700, 3702 extend at least 10 millimeters (“mm”).

Hook 3700 extends along a hook axis and angled jaw portion 3580 extends along a jaw axis. Similarly, Hook 3702 extends along a hook axis and angled jaw portion 3582 extends along a jaw axis. In some instances, the angle between the hook axis and the jaw axis is between 70° and 110°. In some instances, the angle between the hook axis and the jaw axis is between 80° and 100°. In some instances, the angle between the hook axis and the jaw axis is between 85° and 95°. In some instances, the angle between the hook axis and the jaw axis is between 87° and 93°. In some instances, the angle between the hook axis and the jaw axis is between 88° and 89°. In some instances, the angle between the hook axis and the jaw axis is 90°. In some instances, the angle between the hook axis and the jaw axis is greater than 90°.

In addition, Allis forceps 3500 includes engagement members 3594 at an end of the inwardly extending projection. Engagement members 3594 may be used to, for example, grip tissue. Portion 3594A of engagement members 3594 include a plurality of ridges and valleys. Portion 3594B of engagement members 3594 include a plurality of ridges and valleys. The ribs and valleys of portion 3594A mesh with the valleys and ridges of portion 3594B.

Shank 3508 and shank 3510 are releasably coupled at releasable connector 206. Releasable connector 206 allows for the shanks 3508 and 3510 to be readily connected to one another and readily disconnected to one another. The releasable connector 206 is configured such that a member of a surgical staff can readily disconnect and reconnect the shanks 3508, 3510. The term releasable connector does not include permanent connectors that cannot be easily connected and disconnected. Examples of permanent connectors are rivets, screws, bolt/nuts and the like.

Connector 206 is substantially the same as the connectors described above and are not redescribed herein. Connector 206 includes a shaft and retaining members that mate with an opening in the shank.

When Allis forceps 3500 is disassembled, shank 3508 and hook 3590 may be used as a retractor or for other purposes. Similarly, shank 3510 and hook 3592 may be used as a retractor.

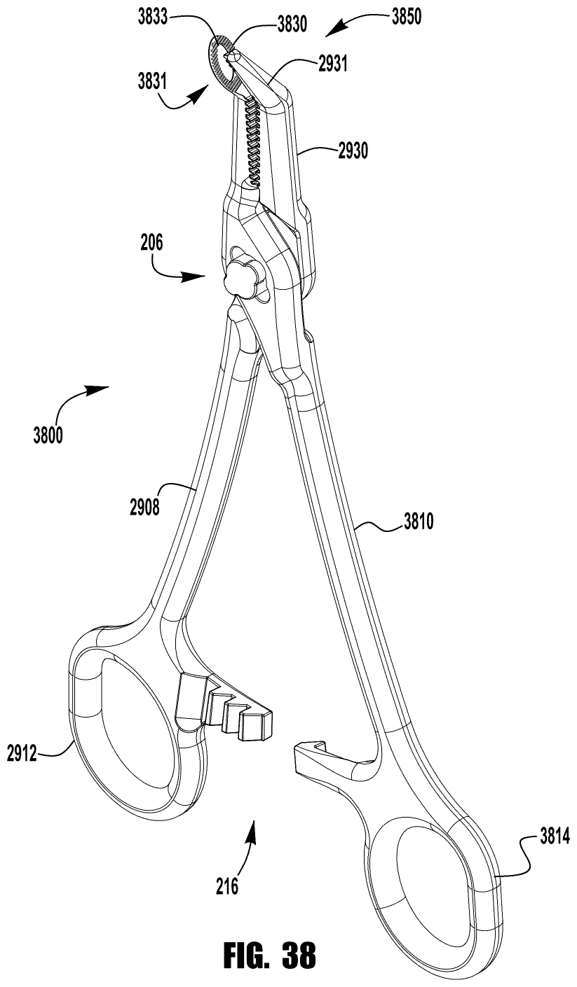

FIG. 38 is an exemplary embodiment of hybrid surgical instrument 3800. Hybrid surgical instruments may be any number of combinations of surgical instruments where one portion of one surgical instrument is combined with a portion of another surgical instruments.

Exemplary hybrid surgical instrument 3800 is a combination of the first shank 2908 of hemostat 2900 and a second shank 3810 of an open ring forceps. Second shank 3810 includes a ring handle 3814 and an open ring 3833 on its working end. Open ring 3833 includes optional ridges 38230. The ridges may increase gripping function of the hybrid surgical instrument. In this exemplary embodiment, open ring 3833 has an oval shape, however, open ring 3833 may have any shape, such as, for example, a circular shape, a rectangular shape, a square shape or the like. The working end 3831 of the open ring 3833 contacts the working end 3850 of shank 2908 of hemostat 2900.

Exemplary hybrid surgical instruments may include, but not limited to, for example, combinations of forceps, hemostats, elevators, trocars, and clamps, combinations of one type of forceps with a second type of forceps, combinations of angled forceps and angled ring forceps, combinations of angled forceps and straight forceps, combinations of a first type of hemostat and a second type of hemostat

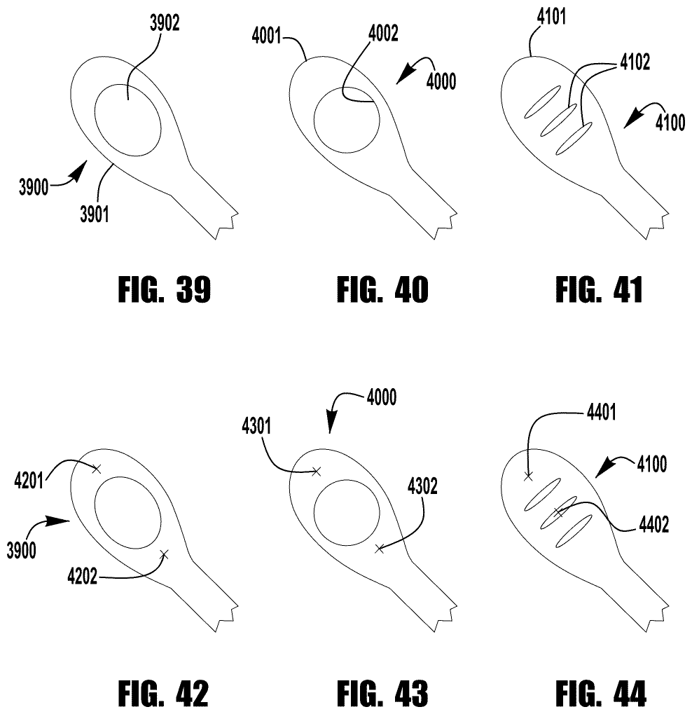

FIG. 39 is an exemplary working end 3900 of a portion of a hybrid surgical instrument. Working end 3900 may be combined with any other type of working end, such as, for example, a forceps, a hemostat, and the like. Exemplary working end 3900 has a circular shape and has an outer portion 3901 and a concave inner portion 3902. Exemplary working end 3900 may have any shape, such as, for example, an oval shape, a rectangular shape, a square shape, a triangle shape, a hexagon shape and the like. The surface may be concave, convex, solid, hollow and the like. Working end 3900 may include ridges (not shown) that extend at least partially across outer portion 3901. FIG. 42 illustrates exemplary contact points 4201 and 4202 that contact the working end (not shown) of a portion of a second surgical instrument, such as, for example, forceps or hemostats.

FIG. 40 is an exemplary working end 4000 of a portion of a hybrid surgical instrument. Working end 4000 may be combined with any other type of working end, such as, for example, a forceps, a hemostat, and the like. Exemplary working end 4000 has a circular shape and has an outer portion 4001 and an open inner portion 4002. Exemplary working end 4000 may have any shape, such as, for example, an oval shape, a rectangular shape, a square shape, a triangle shape, a hexagon shape and the like. The surface may be concave, convex, solid, hollow and the like. Working end 4000 may include ridges (not shown) that extend at least partially across outer portion 4001. FIG. 43 illustrates exemplary contact points 4301 and 4302 that contact the working end (not shown) of a portion of a second surgical instrument, such as, for example, forceps or hemostats.

FIG. 41 is an exemplary working end 4100 of a portion of a hybrid surgical instrument. Working end 4100 may be combined with any other type of working end, such as, for example, a forceps, a hemostat, and the like. Exemplary working end 4100 has a circular shape and has an outer portion 4101 and an inner portion 4102. Exemplary working end 4100 may have any shape, such as, for example, an oval shape, a rectangular shape, a square shape, a triangle shape, a hexagon shape and the like. The surface may be concave, convex, solid, hollow and the like. Working end 4100 includes ridges 4102 that extend at least partially across outer an inner portion of the working end. The ridges may extend entirely across the working end 4100 or partially across. The ridges may extend straight across, may be extend along one or more angles or the like. FIG. 44 illustrates exemplary contact points 4401 and 4402 that contact the working end (not shown) of a portion of a second surgical instrument, such as, for example, forceps or hemostats.

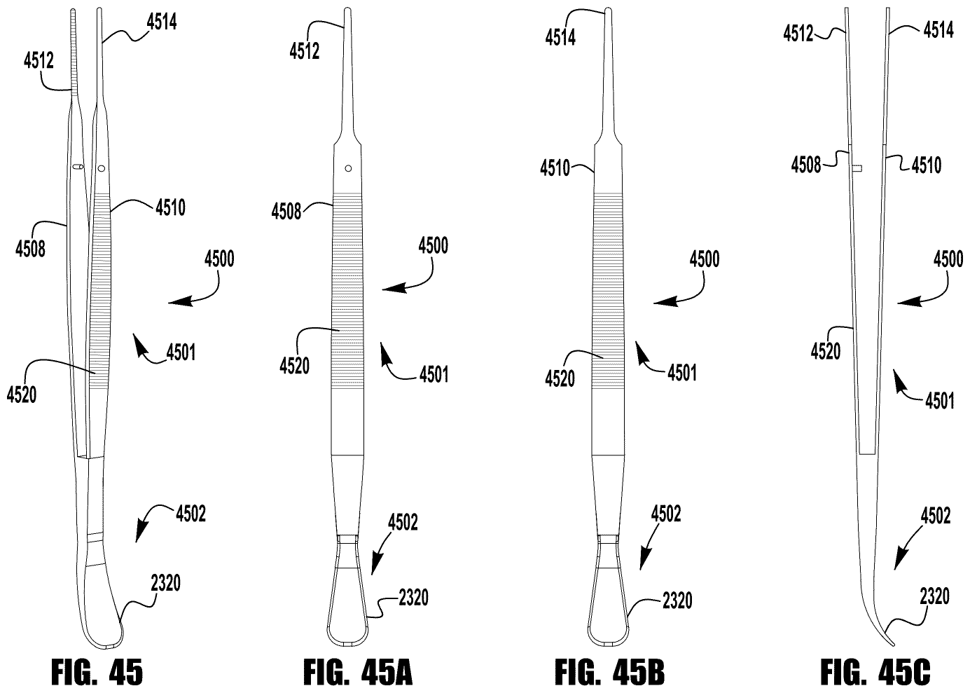

FIGS. 45 through 45C disclose another exemplary embodiment of a multi-functional surgical tool 4500.

FIGS. 45 through 45C illustrate another exemplary multi-function surgical instrument 4500. Multi-function surgical instrument 4500 has components, one on each end, that perform different functions. Multi-function surgical instrument 4500 includes a first portion that is configured as modified Gerald forceps. The Gerald forceps portion 4501 includes a first arm 4508 and a second arm 4510. First a 4508 includes a narrow tip 4512 and second arm 4510 includes a second narrow tip 4514. Optionally, the first narrow tip 4512 and second narrow tip 4514 include one or more teeth (not shown) for gripping items, such as, for example, tissue, dressings, surgical materials and the like. The narrow tips may be straight, curved or angled. The first and second narrow tips 4512 and 4514 operate similar to tweezers and are configured for grasping delicate tissues and/or other objects. The multi-function surgical instrument 4500 may include one or more griping surfaces 4520, which may be a plurality of raised ridges. In addition, the multi-function surgical instrument 4500 includes a spatula retractor/elevator 4502 that is located on a second end of the multi-function surgical instrument 4500 that is opposite of the first and second narrow tips 4512, 4514. The spatula retractor/elevator 4502 may be various shapes, sizes and can be sharp or blunt (different functional edges). Other types of forceps (not shown) may be used in the multi-function surgical instrument in place of the Gerald forceps.

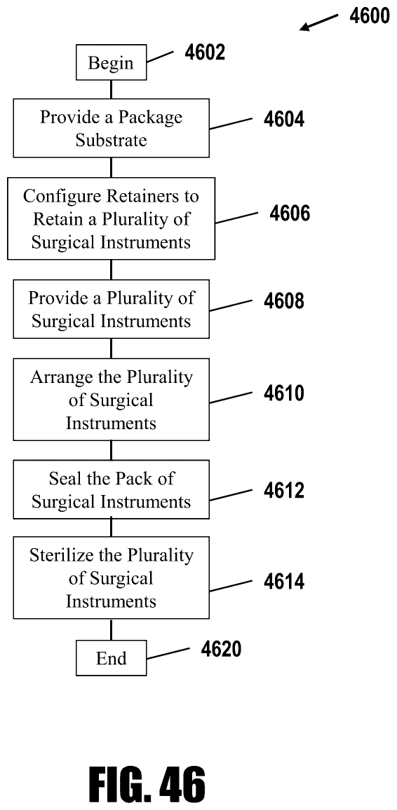

FIG. 46 is an exemplary methodology 4600 for providing a sterilized pack of surgical instruments. The exemplary methodology includes blocks or steps. The exemplary blocks or steps may be performed in different orders than shown and described with exemplary methodology 4600. In some exemplary methodologies, additional blocks or steps may be included. In addition, in some exemplary methodologies, blocks or steps may be eliminated, and/or combined with other blocks or steps.

The exemplary methodology 4600 begins at block 4602. At block 4604 a package substrate is provided. The package substrate may be, for example, a semi-rigid piece of material, such as, for example, cardboard, plastic, or the like. At block 4606, retainers are configured for retaining at least a portion of a plurality of surgical instruments. In some embodiments, the retainers includes a plurality of cut-outs in the substrate. The cut-outs are configured to be bent away from the substrates and are configured to retain at least a portion of a surgical instrument. In some embodiments, the cut-outs include opening that retain portions of surgical instruments.

At block 4608 a plurality of surgical instruments are provided. The plurality of surgical instruments may include, for example, one or more of a multi-functional hemostat 110 and multi-function Allis forceps 120, a multi-function tri-view surgical instrument 160, one or more suction tongue blades 140, ring blade holder 130, a palate retractor 150 and a curette 170. Other surgical instruments may be included and some surgical instruments may be excluded from the package depending on the type of surgical procedure that the surgical pack will be used for. In some embodiments, at least one of the multi-functional hemostat 110 and the multi-functional Allis forceps 120 is made of two parts and the two parts are readily separable from one another. In some embodiments the surgical set of instruments may be an expandable set of surgical instruments. For example, selected surgical instruments are configured to function as multiple types of instruments, e.g. the forceps may be used as forceps or may be disassembled and used as, for example, one or more retractors. Selected surgical instruments may be disassembled and combined with parts of one or more other disassembled surgical instruments. These combined parts may be referred to herein as hybrid surgical instruments. The hybrid surgical instruments may have jaw portions or different working end portions. The jaw portions or working end portions of the hybrid surgical instruments have different configurations. In addition, selected surgical instruments are multi-functional and perform the functions of multiple prior art surgical instruments. These surgical instruments may also be referred to herein as hybrid surgical instruments or multi-function surgical instruments.

The surgical instruments are arranged at block 4610. In some embodiments, packaging space is saved by arranging surgical instruments used earlier in the desired surgery to be placed on or above portions of later uses surgical instruments. In addition, such overlap places the “next up” surgical instrument in a ready position. Thus, package may provide a presorted set of surgical instruments so an operator or surgeon can “open and operate.” The package configuration provides a ready to use set of surgical instruments that are preselected and may be arranged for a selected type of surgery.

An exemplary arrangement of the plurality of surgical instruments includes hemostat 110 that is arranged on top of Allis forceps 1120. In addition, multi-functional tri-view surgical instrument 160 is placed in package. Suction tongue blade 140 is placed the package and extends over multi-functional tri-view surgical instrument 160. Palate retractor is placed in the package and ring blade holder 130 is place in the package. This allows the surgical instruments to be removed from package in the order that they are needed. Ring blade holder 130 is typically removed first. Suction tongue blade 140 is removed next and secured to ring blade holder 130 to form a mouth gag. The mouth gag is connected to a suction hose (not shown) and placed in use. Hemostat 110 is removed. Palate retractor 150 is removed and placed in use with the hemostat 110. Multi-functional tri-view surgical instrument 160 and curette 170 may then be removed when needed. In addition, the Allis forceps 120 may be removed and placed into use.

The pack of surgical instruments are sealed in an outer sanitary wrap or plastic outer wrap, such as, for example a Tyvek® wrap, at block 4612 and the methodology ends at block 4620. At block 4614 the plurality of surgical instruments are sterilized. Preferably, the plurality of surgical instruments are arranged in and secured to the package. Preferably the retainers are configured to allow sterilization material to flow around and contact all surfaces of the surgical instruments. In some cases, the sterilization process may occur prior to the surgical instruments being arranged in the package.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. It is not the intention of the applicant to restrict or in any way limit the scope of the appended claims to such detail. Unless expressly excluded herein, all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order in which the steps are presented to be construed as required or necessary unless expressly so stated.

1. A two-part surgical instrument comprising:
a first shank;
the first shank having a first ring handle at a first end, a first jaw portion on the other end, and a shaft and one or more retainers;
a second shank;
the second shank having a second ring handle at a first end and a second jaw portion on the other end and an opening configured to receive the shaft;
wherein the first shank is connected to the second shank by sliding the opening of the second shank over the shaft of the first shank and the first shank is disconnected from the second shank by pulling the second shank away from the first shank.

2. The two-part surgical instrument of claim 1 wherein the second shank comprises one or more second portions of the opening and wherein the one or more retainers are configured to fit through the one or more second portions of the opening when the one or retainers are aligned with the one or more second portions of the opening.

3. The two-part surgical instrument of claim 1 wherein the one or more retainers comprises one or more lobes.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The two-part surgical instrument of claim 1 wherein the first shank and the second shank are separated by aligning the one or more retaining members with an opening and moving the first shank away from the second shank.

16. The two-part surgical instrument of any of claim 1 wherein the first shank and the second shank must be orientated at an opening angle of greater than 90 degrees to separate the first shank from the second shank.

17. The two-part surgical instrument of claim 1 wherein the two-part surgical instrument is non-metallic.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A palate retractor comprising:
a body;
a mouth gag connection member on a first end of the body;
a grip member;
a palate engagement member located on a second end of the body; and
a pair of wings extending outward from the body proximate the palate engagement member.

39. (canceled)

40. (canceled)

41. (canceled)

42. (canceled)

43. The palate retractor of claim 38 wherein the grip member further comprises an arcuate rib.

44. (canceled)

45. (canceled)

46. The palate retractor of claim 38 wherein the palate engagement member is curved.

47. (canceled)

48. (canceled)

49. (canceled)

50. (canceled)

51. The palate retractor of claim 38 wherein the palate engagement member curves upward and the pair of wings curve laterally.

52. (canceled)

53. (canceled)

54. (canceled)

55. (canceled)

56. (canceled)

57. A preorganized pack of surgical instruments comprising:
a package;
at least one hemostat;
at least one forceps;
wherein one of the hemostat and the forceps are readily separatable into two pieces;
wherein the at least one hemostat and the at least one forceps are devoid of rivets, screws or bolts.

58. The preorganized pack of surgical instruments of claim 57 wherein the package comprises:
a semi-rigid material;
plurality of tabs;
the plurality of tabs being cut out from a portion of the semi-rigid material;
one or more openings in the plurality of tabs;
wherein the plurality of tabs are bent away from the semi-rigid material and the one or more openings are configured to secure at least a portion of a surgical instrument.

59. The preorganized pack of surgical instruments of claim 58 wherein the plurality of tabs are configured such that a first surgical instrument is secured by one or more openings; and a second surgical instrument is secured by one or more different openings; and
wherein at least a portion of the second surgical instrument is located above the first surgical instrument.

60. (canceled)

61. (canceled)

62. (canceled)

63. (canceled)

64. (canceled)

65. The preorganized pack of surgical instruments of claim 57 comprises:
a hemostat comprising a first shank and a second shank connectable by a releasable connector;
a forceps comprising a first shank and a second shank connectable by a releasable connector; and
a multi-functional surgical instrument comprising at least two of a mirror, a spatula and a retractor.

66. The preorganized pack of surgical instruments of claim 65 further comprising:
a ring blade holder and a suction tongue blade.

67. The preorganized pack of surgical instruments of claim 66 wherein the suction tongue blade has a suction inlet; and
wherein the suction inlet is located along a centerline of the suction tongue blade.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A surgical instrument comprising:
a first part having a first shank, a first ring handle, a first working end and a first portion of a releasable connector; and
a second part having a second shank, a second ring handle, a second working end and a second prat of a releasable connector;
wherein the first working end has a different configuration than the second working end;
wherein the first portion of a releasable connector connects to the second portion of a releasable connector to couple the first part to the second part in a coupled position;
wherein in the coupled position, movement of the first ring handle away from the second ring handle causes the first working end and the second working end to move away from one another; and
wherein movement of the first ring handle toward the second ring handle causes the first working end to move toward the second working end.

87. The surgical instrument of claim 86 wherein the first portion of the releasable connector comprises one of an opening and a retainer.

88. The surgical instrument of claim 87 wherein the second portion of the releasable connector comprises the other of the opening and the retainer.

89. The surgical instrument of claim 86 wherein the different configuration comprises a first working end that is a first type of forceps and the second working end is a second type of forceps.

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)
